SEQUENCE LISTING (11 sequences)

SEQ ID NO: 1 - protein, 31 aa, synthetic construct
  VARIANT 1: note = H or Y
  VARIANT 2: note = A, Aib or Aib-psi[CH2NH]
  VARIANT 13: note = Y or Aib
  VARIANT 28: note = K or R
  VARIANT 30: note = R or Dpr
XXEGTFTSDVSSXLEGQAAKEFIAWLVXGXG

SEQ ID NO: 2 - protein, 38 aa, synthetic construct
  VARIANT 1: note = H or Y
  VARIANT 2: note = A, Aib or Aib-psi[CH2NH]
  VARIANT 13: note = Y or Aib
  VARIANT 14: note = L or Aib
  VARIANT 28: note = K, R or G
  VARIANT 30: note = R or Dpr
  VARIANT 31: note = G or S
XXEGTFTSDVSSXXEGQAAKEFIAWLVXGXXSGAPPPS

SEQ ID NO: 3 - protein, 31 aa, synthetic construct
  SITE 2: note = Aib
  SITE 13: note = Aib
  MOD_RES 20: note = epsilon-amino group is substituted with a 2-[2-(2-aminoethoxy)ethoxy]acetic acid-2-[2-(2-aminoethoxy)ethoxy]acetic acid-gamma-E-(CH2)m-OH wherein m is 18
YXEGTFTSDVSSXLEGQAAKEFIAWLVRGRG

SEQ ID NO: 4 - protein, 31 aa, synthetic construct
  MOD_RES 2: note = Aib-psi[CH2NH]
  SITE 13: note = Aib
  MOD_RES 20: note = epsilon-amino group is substituted with a 2-[2-(2-aminoethoxy)ethoxy]acetic acid-2-[2-(2-aminoethoxy)ethoxy]acetic acid-gamma-E-(CH2)m-OH wherein m is 18
HXEGTFTSDVSSXLEGQAAKEFIAWLVRGRG

SEQ ID NO: 5 - protein, 31 aa, synthetic construct; identical to SEQ ID NO: 4
  SITE 2: note = Aib
  SITE 13: note = Aib
  MOD_RES 20: note = epsilon-amino group is substituted with a 2-[2-(2-aminoethoxy)ethoxy]acetic acid-2-[2-(2-aminoethoxy)ethoxy]acetic acid-gamma-E-(CH2)m-OH wherein m is 18

SEQ ID NO: 6 - protein, 31 aa, synthetic construct
  SITE 2: note = Aib
  SITE 13: note = Aib
  MOD_RES 20: note = epsilon-amino group is substituted with a 2-[2-(2-aminoethoxy)ethoxy]acetic acid-2-[2-(2-aminoethoxy)ethoxy]acetic acid-gamma-E-(CH2)m-OH wherein m is 18
  SITE 30: note = Dpr
HXEGTFTSDVSSXLEGQAAKEFIAWLVRGXG

SEQ ID NO: 7 - protein, 38 aa, synthetic construct
  SITE 2: note = Aib
  SITE 14: note = Aib
  MOD_RES 20: note = epsilon-amino group is substituted with a 2-[2-(2-aminoethoxy)ethoxy]acetic acid-2-[2-(2-aminoethoxy)ethoxy]acetic acid-gamma-E-(CH2)m-OH wherein m is 18
  SITE 38: note = C-terminus is amidated
HXEGTFTSDVSSYXEGQAAKEFIAWLVGGPSSGAPPPS

SEQ ID NO: 8 - protein, 30 aa, synthetic construct
HAEGTFTSDVSSYLEGQAAKEFIAWLVKGR

SEQ ID NO: 9 - protein, 39 aa, synthetic construct
HGEGTFTSDLSKQMEEEAVRLFIEWLKNGGPSSGAPPPS

SEQ ID NO: 10 - protein, 4 aa, synthetic construct
RGRG

SEQ ID NO: 11 - protein, 11 aa, synthetic construct
  SITE 11: note = C-terminus is amidated
GGPSSGAPPPS

(19) United States
(12) Patent Application Publication
(10) Pub. No.: US 2025/0074964 A1
(43) Pub. Date: Mar. 6, 2025

(54) STABLE GLP-1 ANALOGS FOR THE TREATMENT OF HUMAN DISEASE AND DISORDERS

(71) Applicant: Ambio, Inc., Grovetown, GA (US)

(72) Inventors: Juncai BAI, Jacksonville, FL (US); Guoqing ZHANG, Shanghai (CN); Fuliang ZHANG, Shanghai (CN); Michael William PENNINGTON, Sapphire, NC (US); Simrat P. SINGH, Evans, GA (US)

(21) Appl. No.: 18/825,825

(22) Filed: Sep. 5, 2024

(30) Foreign Application Priority Data
Sep. 6, 2023 (WO) PCT/CN2023/117248

(51) Int. Cl. C07K 14/605; A61K 38/00; A61K 38/26; A61K 45/06; A61P 3/04

(52) U.S. Cl. CPC C07K 14/605; A61K 38/26; A61K 45/06; A61P 3/04; A61K 38/00

(57) ABSTRACT

The disclosure described herein provides for enzymatically stable and potent GLP-1 analogs (i.e., derivatives) for the treatment of metabolic disease or disorder in a subject. In some embodiments, the GLP-1 analog is a derivatized GLP-1 peptide, wherein the derivatized peptide has 1, 2, 3, 4, 5, 6, 7, 8, 9, or 10 amino acid differences from any one of GLP-1, GLP-1(7-39), GLP-1(7-36), GLP-1(7-37). In certain embodiments, the GLP-1 analog comprises at least one lysine substituent. The GLP-1 analogs described herein demonstrate potent receptor binding and extended plasma half-life. A pharmaceutical composition of the GLP-1 analogs is described herein for the treatment of metabolic disease or disorder in a subject, such as diabetes or obesity.

### RELATED APPLICATIONS

This application claims priority to, and the benefit of, International Application PCT/CN2023/117248, filed on Sep. 6, 2023. The contents of this application are incorporated herein by reference in its entirety.

### REFERENCE TO AN ELECTRONIC SEQUENCE LISTING

The contents of the electronic sequence listing (AMBO_001_001US_SeqList_ST26.xml; Size: 22,243 bytes; and Date of Creation: Aug. 21, 2024) are herein incorporated by reference in its entirety.

### BACKGROUND

GLP-1 is a product of the proglucagon, a member of the secretin-VIP family of peptides, and is an important gut hormone with regulatory function in glucose metabolism and gastrointestinal secretion and metabolism. The amino acid sequence of GLP-1 is given by Schmidt et al. (Diabetologia 28 704-707 (1985). Human GLP-1 is a 37 amino acid residue peptide originating from preproglucagon which is synthesized in the L-cells in the distal ileum, in the pancreas, and in the brain. Processing of preproglucagon to GLP-1 (7-36)amide, GLP-1 (7-37) and GLP-2 occurs mainly in the L-cells.

GLP-1 is effective in stimulating insulin secretion in diabetic patients. In addition, and in contrast to the other insulinotropic hormones (perhaps with the exception of secretin) it also potently inhibits glucagon secretion. GLP-1 has pronounced blood glucose-lowering effects, particularly in patients with diabetes. Because of the glucose dependency of the insulinotropic and glucagonostatic actions, the glucose-lowering effect is self-limiting, and the hormone does not cause hypoglycemia regardless of dose. The effects are preserved in patients with diabetes mellitus, in whom infusions of slightly supraphysiological doses of GLP-1 may completely normalize blood glucose values in spite of poor metabolic control and secondary failure to sulphonylurea.

In addition to its effects on pancreatic islets, GLP-1 has powerful actions on the gastrointestinal tract. Infused in physiological amounts GLP-1 potently inhibits pentagastrin-induced and meal-induced gastric acid secretion. It also inhibits gastric emptying rate and pancreatic enzyme secretion.

The GLP-1 peptide is fully active after subcutaneous administration but is rapidly degraded mainly due to degradation by dipeptidyl peptidase IV-like enzymes. The plasma half-life of GLP-1 in humans is 2 minutes. Limited half-lives of GLP-1 and current analogs lead to increased injection frequency for the patient. There is a particular need to develop GLP-1 analogs that are more stable against enzyme degradation, but with enhanced or maintained potency, in order to allow for less frequent patient injections due to a prolonged half-life.

### SUMMARY OF THE INVENTION

The disclosure described herein provides for enzymatically stable and potent GLP-1 analogs (i.e., derivatives) for the treatment of metabolic disease or disorder in a subject. In some embodiments, the GLP-1 analog is a derivatized GLP-1 peptide, wherein the derivatized peptide has 1, 2, 3, 4, 5, 6, 7, 8, 9, or 10 amino acid differences from any one of GLP-1, GLP-1(7-39), GLP-1(7-36), GLP-1(7-37). In some embodiments, the GLP-1 analog is a derivatized GLP-1 peptide, wherein the derivatized peptide has greater than a 10 amino acid difference from any one of GLP-1, GLP-1(7-39), GLP-1(7-36), GLP-1(7-37). In certain embodiments, the GLP-1 analog comprises at least one lysine substituent. The GLP-1 analogs described herein demonstrate potent receptor binding and extended plasma half-life. A pharmaceutical composition of the GLP-1 analogs is described herein for the treatment of metabolic disease or disorder in a subject, such as diabetes or obesity.

### BRIEF DESCRIPTION OF THE DRAWING(S)

FIG. 1 illustrates the binding affinity of analog peptides to GLP-1R as determined by a luciferase reporter assay.

FIGS. 2A-2B illustrate the binding affinity of analog peptides to GLP-1R as determined by a luciferase reporter assay with the error indicated. FIG. 2A depicts the percentage of inhibition of the analog peptides to GLP-1R compared to semaglutide and liraglutide. FIG. 2B depicts the GLP-1R activation EC50 of the analog peptides compared to semaglutide and liraglutide.

FIG. 3 illustrates the GLP-1 analogs.

FIG. 4 illustrates native GLP-1(7-36) sequence.

FIG. 5 illustrates the exendin-4 sequence.

FIGS. 6A-6B illustrate the binding affinity of analog peptides to GLP-1R as determined by a Tag-Lite® Receptor Binding FRET Assay in the absence of HSA. FIG. 6A depicts the percentage of inhibition of the analog peptides to the GLP-1R compared to semaglutide and liraglutide. FIG. 6B depicts the GLP-1R activation IC50 of the analog peptides compared to semaglutide and liraglutide.

FIGS. 7A-7B illustrate the binding affinity of analog peptides to GLP-1R as determined by a Tag-Lite® Receptor Binding FRET Assay in the presence of 2% HSA. FIG. 7A depicts the percentage of inhibition of the analog peptides to the GLP-1R compared to semaglutide and liraglutide. FIG. 7B depicts the GLP-1R activation IC50 of the analog peptides compared to semaglutide and liraglutide.

FIGS. 8A-8B illustrate the binding affinity of analog peptides to GLP-1R as determined by a Tag-Lite® Receptor Binding FRET Assay in the presence of 2% HSA compared to the absence of HSA. FIG. 8A shows the fold increase in IC50 of analog peptides to GLP-1R in the presence of 2% HSA compared to the absence of HSA. FIG. 8B shows the activity change in the analog peptides to GLP-1R in the presence and absence of HSA.

FIG. 9 illustrates the interaction of analog peptides to GLP-1R with glucagon receptor as determined by cAMP production in a HEK293/GCGR/Gα15 cell-based assay compared to glucagon as a positive control.

FIG. 10 illustrates the stability of semaglutide formulation at a concentration of 1 mg/mL after incubation under a variety of conditions. Purity was measured using HPLC and compared to a freshly prepared formation where the purity was measured immediately following preparation (Fresh). “A” represents a formulation incubated at −80° C. for 7 days. “B” represents a formulation incubated at 4° C. for 1 day. “C” represents a formulation incubated at 4° C. for 3 days. “D” represents a formulation incubated at 4° C. for 7 days.

FIG. 11 illustrates the stability of APi3709 formulation at a concentration of 1 mg/mL after incubation under a variety of conditions. Purity was measured using HPLC and compared to a freshly prepared formation where the purity was measured immediately following preparation (Fresh). “A” represents a formulation incubated at −80° C. for 7 days. “B” represents a formulation incubated at 4° C. for 1 day. “C” represents a formulation incubated at 4° C. for 3 days. “D” represents a formulation incubated at 4° C. for 7 days.

FIG. 12 illustrates the stability of APi3710 formulation at a concentration of 1 mg/mL after incubation under a variety of conditions. Purity was measured using HPLC and compared to a freshly prepared formation where the purity was measured immediately following preparation (Fresh). “A” represents a formulation incubated at −80° C. for 7 days. “B” represents a formulation incubated at 4° C. for 1 day. “C” represents a formulation incubated at 4° C. for 3 days. “D” represents a formulation incubated at 4° C. for 7 days.

FIG. 13 illustrates the stability of APi3711 formulation at a concentration of 1 mg/mL after incubation under a variety of conditions. Purity was measured using HPLC and compared to a freshly prepared formation where the purity was measured immediately following preparation (Fresh). “A” represents a formulation incubated at −80° C. for 7 days. “B” represents a formulation incubated at 4° C. for 1 day. “C” represents a formulation incubated at 4° C. for 3 days. “D” represents a formulation incubated at 4° C. for 7 days.

FIG. 14 illustrates the stability of APi3712 formulation at a concentration of 1 mg/mL after incubation under a variety of conditions. Purity was measured using HPLC and compared to a freshly prepared formation where the purity was measured immediately following preparation (Fresh). “A” represents a formulation incubated at −80° C. for 7 days. “B” represents a formulation incubated at 4° C. for 1 day. “C” represents a formulation incubated at 4° C. for 3 days. “D” represents a formulation incubated at 4° C. for 7 days.

FIG. 15 illustrates the stability of APi3713 formulation at a concentration of 1 mg/mL after incubation under a variety of conditions. Purity was measured using HPLC and compared to a freshly prepared formation where the purity was measured immediately following preparation (Fresh). “A” represents a formulation incubated at −80° C. for 7 days. “B” represents a formulation incubated at 4° C. for 1 day. “C” represents a formulation incubated at 4° C. for 3 days. “D” represents a formulation incubated at 4° C. for 7 days.

FIGS. 16A-16B illustrate the body weight changes of a diet-induced obesity (DIO) mouse model treated with analog peptides to GLP-1R. FIG. 16A shows the body weight changes of DIO mice treated with analog peptides APi3709, APi3710, APi3711, APi3712, and APi3713 compared to untreated mice (DIO vehicle) and DIO mice treated with semaglutide. FIG. 16B shows the body weight changes±standard error of the mean (SEM) of DIO mice treated with analog peptide APi3712 compared to untreated mice (DIO vehicle) and DIO mice treated with semaglutide.

FIG. 17 illustrates the body weight changes of a diet-induced obesity (DIO) mouse model treated with analog peptides to GLP-1R.

FIGS. 18A-18B illustrate the food consumption of a diet-induced obesity (DIO) mouse model treated with analog peptides to GLP-1R. FIG. 18A shows the food consumption of DIO mice treated with analog peptides APi3709, APi3710, APi3711, APi3712, and APi3713 compared to untreated mice (DIO vehicle) and DIO mice treated with semaglutide. FIG. 18B shows the food consumption of DIO mice treated with analog peptide APi3711, APi3712, and APi3713 compared to untreated mice (DIO vehicle) and DIO mice treated with semaglutide.

FIGS. 19A-19B illustrates the non-fasted blood glucose of a diet-induced obesity (DIO) mouse model treated with analog peptides to GLP-1R. FIG. 19A shows the non-fasted blood glucose of DIO mice treated with analog peptides APi3709, APi3710, APi3711, APi3712, and APi3713 compared to untreated mice (DIO vehicle) and DIO mice treated with semaglutide. FIG. 19B shows the non-fasted blood glucose±standard error of the mean (SEM) of DIO mice treated with analog peptide APi3712 compared to untreated mice (DIO vehicle) and DIO mice treated with semaglutide.

FIG. 20 illustrates the plasma insulin level of a diet-induced obesity (DIO) mouse model treated with analog peptides to GLP-1R.

FIG. 21 illustrates the plasma insulin level of a diet-induced obesity (DIO) mouse model treated with analog peptides to GLP-1R.

FIG. 22 illustrates the c-peptide plasma concentration of a diet-induced obesity (DIO) mouse model treated with analog peptides to GLP-1R.

FIG. 23 illustrates the c-peptide plasma concentration of a diet-induced obesity (DIO) mouse model treated with analog peptides to GLP-1R.

FIG. 24 illustrates the binding affinity of analog peptides APi3712 and APi3713 compared to tirzepatide to GIPR as determined by a cell-based human GIPR luciferase reporter assay.

### DETAILED DESCRIPTION OF THE INVENTION

A simple system is used to describe fragments and analogs of GLP-1. For example, Gly⁸-GLP-1(7-37) designates a peptide that relates to GLP-1 by the deletion of the amino acid residues at positions 1 to 6 and substituting the naturally occurring amino acid residue in position 8 (Ala) by Gly. Similarly, as an example, Lys³⁴(N^{ε}-tetradecanoyl)-GLP-1(7-37) designates GLP-1 (7-37) wherein the ε-amino group of the Lys residue in position 34 has been tetradecanoylated.

The term “an analog” is defined herein as a peptide wherein one or more amino acid residues of the parent GLP-1 peptide have been substituted by another amino acid residue. In some embodiments, the analog comprises a C-terminal or N-terminal extension. In some embodiments, the present disclosure relates to a GLP-1 analog wherein the parent peptide is GLP-1(1-45) or an analog thereof. In some embodiments, the parent peptide is GLP-1(1-35), GLP-1 (1-36), GLP-1(1-36)amide, GLP-1(1-37), GLP-1(1-38), GLP-1(1-39), GLP-1(1-40), GLP-1(1-41) or an analog thereof.

In some embodiments, the GLP-1 analog comprises at least one 2-aminoisobutyric acid (Aib). In some embodiments, the GLP-1 analog comprises at least one Aib-ψ[CH₂NH]-Xaa, wherein there is a reduced peptide bond (i.e., CH₂NH) between the Aib group and Xaa. In some embodiments, the GLP-1 analog comprises at least one L-2,3-diaminopropionic acid.

In some embodiments, the GLP-1 analog is Xaa7-Xaa8-Xaa9-Xaa10-Xa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31-Xaa32-Xaa33-Xaa34-Xaa35-Xaa36-Xaa37 (SEQ ID NO: 1), wherein Xaa7 is His or Tyr, Xaa8 is Ala or Aib or Aib-ψ[CH₂NH], Xaa9 is Glu, Xaa10 is Gly, Xaa11 is Thr, Xaa12 is Phe, Xaa13 is Thr, Xaa14 is Ser, Xaa15 is Asp, Xaa16 is Val, Xaa17 is Ser, Xaa18 is Ser, Xaa19 is Tyr or Aib, Xaa20 is Leu, Xaa21 is Glu, Xaa22 is Gly, Xaa23 is Gln, Xaa24 is Ala, Xaa25 is Ala, Xaa26 is Lys, Xaa27 is Glu, Xaa28 is Phe, Xaa29 is Ile, Xaa30 is Ala, Xaa31 is Trp, Xaa32 is Leu, Xaa33 is Val, Xaa34 is Lys or Arg, Xaa35 is Gly, Xaa36 is Arg or DAP (L-2,3-diaminopropionic acid), Xaa37 is Gly.

In some embodiments, the GLP-1 analog is Xaa7-Xaa8-Xaa9-Xaa10-Xa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31-Xaa32-Xaa33-Xaa34-Xaa35-Xaa36-Xaa37-Xaa38-Xaa39-Xaa40-Xaa4l-Xaa42-Xaa43-Xaa44 (SEQ ID NO: 2), wherein Xaa7 is His or Tyr, Xaa8 is Ala or Aib or Aib-ψ[CH₂NH], Xaa9 is Glu, Xaa10 is Gly, Xaa11 is Thr, Xaa12 is Phe, Xaa13 is Thr, Xaa14 is Ser, Xaa15 is Asp, Xaa16 is Val, Xaa17 is Ser, Xaa18 is Ser, Xaa19 is Tyr or Aib, Xaa20 is Leu or Aib, Xaa21 is Glu, Xaa22 is Gly, Xaa23 is Gln, Xaa24 is Ala, Xaa25 is Ala, Xaa26 is Lys, Xaa27 is Glu, Xaa28 is Phe, Xaa29 is Ile, Xaa30 is Ala, Xaa31 is Trp, Xaa32 is Leu, Xaa33 is Val, Xaa34 is Lys or Arg or Gly, Xaa35 is Gly, Xaa36 is Arg or DAP (L-2,3-diaminopropionic acid) or Pro, Xaa37 is Gly or Ser, Xaa38 is Ser, Xaa39 is Gly, Xaa40 is Ala, Xaa41 is Pro, Xaa42 is Pro, Xaa43 is Pro, Xaa44 is Ser.

In some embodiments, the GLP-1 analog comprises a substituent on a Lys. In some embodiments, the ε-amino group Lys is substituted. In some embodiments, the ε-amino group Lys at position 26 of the GLP-1 analog is substituted. In some embodiments, the ε-amino group Lys at position 26 of the GLP-1 analog is substituted with a lipophilic substituent. In some embodiments, the ε-amino group Lys at position 26 of the GLP-1 analog is substituted with a lipophilic substituent via a spacer.

In some embodiments, the ε-amino group of Lys is substituted with a AEEA-AEEA-γ-Glu-(CH₂)ₘ—OH (AEEA=2-[2-(2-aminoethoxy)ethoxy]acetic acid) wherein m is 12, 13, 14, 15, 16, 17, 18, 19, 20, 21, or 22. In some embodiments, the ε-amino group of Lys is substituted with an AEEA-AEEA-γ-Glu-(CH₂)ₘ—OH (AEEA=2-[2-(2-aminoethoxy)ethoxy]acetic acid) wherein m is 18 as shown in the following structure:

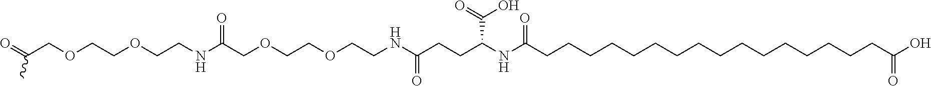

In some embodiments, the GLP-1 analog is referred to as APi3709. APi3709 is Xaa7-Xaa8-Xaa9-Xaa10-Xa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31-Xaa32-Xaa33-Xaa34-Xaa35-Xaa36-Xaa37 (SEQ ID NO: 3), wherein Xaa7 is Tyr, Xaa8 is Aib, Xaa9 is Glu, Xaa10 is Gly, Xaa11 is Thr, Xaa12 is Phe, Xaa13 is Thr, Xaa14 is Ser, Xaa15 is Asp, Xaa16 is Val, Xaa17 is Ser, Xaa18 is Ser, Xaa19 is Aib, Xaa20 is Leu, Xaa21 is Glu, Xaa22 is Gly, Xaa23 is Gln, Xaa24 is Ala, Xaa25 is Ala, Xaa26 is Lys, Xaa27 is Glu, Xaa28 is Phe, Xaa29 is Ile, Xaa30 is Ala, Xaa31 is Trp, Xaa32 is Leu, Xaa33 is Val, Xaa34 is Arg, Xaa35 is Gly, Xaa36 is Arg, Xaa37 is Gly, wherein the ε-amino group of Lys26 is substituted with a AEEA-AEEA-γ-Glu-(CH₂)ₘ—OH (AEEA=2-[2-(2-aminoethoxy)ethoxy]acetic acid) wherein m is 18.

In some embodiments, the GLP-1 analog is referred to as APi3710. APi3710 is Xaa7-Xaa8-Xaa9-Xaa10-Xa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31-Xaa32-Xaa33-Xaa34-Xaa35-Xaa36-Xaa37 (SEQ ID NO: 4), wherein Xaa7 is His, Xaa8 is Aib-ψ[CH₂NH], Xaa9 is Glu, Xaa10 is Gly, Xaa11 is Thr, Xaa12 is Phe, Xaa13 is Thr, Xaa14 is Ser, Xaa15 is Asp, Xaa16 is Val, Xaa17 is Ser, Xaa18 is Ser, Xaa19 is Aib, Xaa20 is Leu, Xaa21 is Glu, Xaa22 is Gly, Xaa23 is Gln, Xaa24 is Ala, Xaa25 is Ala, Xaa26 is Lys, Xaa27 is Glu, Xaa28 is Phe, Xaa29 is Ile, Xaa30 is Ala, Xaa31 is Trp, Xaa32 is Leu, Xaa33 is Val, Xaa34 is Arg, Xaa35 is Gly, Xaa36 is Arg, Xaa37 is Gly wherein the ε-amino group of Lys26 is substituted with a AEEA-AEEA-γ-Glu-(CH₂)ₘ—OH (AEEA=2-[2-(2-aminoethoxy)ethoxy]acetic acid) wherein m is 18.

In some embodiments, the GLP-1 analog is referred to as APi3711. APi3711 is Xaa7-Xaa8-Xaa9-Xaa10-Xa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31-Xaa32-Xaa33-Xaa34-Xaa35-Xaa36-Xaa37 (SEQ ID NO: 5), wherein Xaa7 is His, Xaa8 is Aib, Xaa9 is Glu, Xaa10 is Gly, Xaa11 is Thr, Xaa12 is Phe, Xaa13 is Thr, Xaa14 is Ser, Xaa15 is Asp, Xaa16 is Val, Xaa17 is Ser, Xaa18 is Ser, Xaa19 is Aib, Xaa20 is Leu, Xaa21 is Glu, Xaa22 is Gly, Xaa23 is Gln, Xaa24 is Ala, Xaa25 is Ala, Xaa26 is Lys, Xaa27 is Glu, Xaa28 is Phe, Xaa29 is Ile, Xaa30 is Ala, Xaa31 is Trp, Xaa32 is Leu, Xaa33 is Val, Xaa34 is Arg, Xaa35 is Gly, Xaa36 is Arg, Xaa37 is Gly wherein the ε-amino group of Lys26 is substituted with a AEEA-AEEA-γ-Glu-(CH₂)ₘ—OH (AEEA=2-[2-(2-aminoethoxy)ethoxy]acetic acid) wherein m is 18.

In some embodiments, the GLP-1 analog is referred to as APi3712. APi3712 is Xaa7-Xaa8-Xaa9-Xaa10-Xa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31-Xaa32-Xaa33-Xaa34-Xaa35-Xaa36-Xaa37 (SEQ ID NO: 6), wherein Xaa7 is His, Xaa8 is Aib, Xaa9 is Glu, Xaa10 is Gly, Xaa11 is Thr, Xaa12 is Phe, Xaa13 is Thr, Xaa14 is Ser, Xaa15 is Asp, Xaa16 is Val, Xaa17 is Ser, Xaa18 is Ser, Xaa19 is Aib, Xaa20 is Leu, Xaa21 is Glu, Xaa22 is Gly, Xaa23 is Gln, Xaa24 is Ala, Xaa25 is Ala, Xaa26 is Lys, Xaa27 is Glu, Xaa28 is Phe, Xaa29 is Ile, Xaa30 is Ala, Xaa31 is Trp, Xaa32 is Leu, Xaa33 is Val, Xaa34 is Arg, Xaa35 is Gly, Xaa36 is DAP (L-2,3-diaminopropionic acid), Xaa37 is Gly wherein the ε-amino group of Lys26 is substituted with a AEEA-AEEA-γ-Glu-(CH₂)ₘ—OH (AEEA=2-[2-(2-aminoethoxy)ethoxy]acetic acid) wherein m is 18.

In some embodiments, the GLP-1 analog is referred to as APi3713. APi3713 is Xaa7-Xaa8-Xaa9-Xaa10-Xa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31-Xaa32-Xaa33-Xaa34-Xaa35-Xaa36-Xaa37-Xaa38-Xaa39-Xaa40-Xaa41-Xaa42-Xaa43-Xaa44 (SEQ ID NO: 7), wherein Xaa7 is His, Xaa8 is Aib, Xaa9 is Glu, Xaa10 is Gly, Xaa11 is Thr, Xaa12 is Phe, Xaa13 is Thr, Xaa14 is Ser, Xaa15 is Asp, Xaa16 is Val, Xaa17 is Ser, Xaa18 is Ser, Xaa19 is Tyr, Xaa20 is Aib, Xaa21 is Glu, Xaa22 is Gly, Xaa23 is Gln, Xaa24 is Ala, Xaa25 is Ala, Xaa26 is Lys, Xaa27 is Glu, Xaa28 is Phe, Xaa29 is Ile, Xaa30 is Ala, Xaa31 is Trp, Xaa32 is Leu, Xaa33 is Val, Xaa34 is Gly, Xaa35 is Gly, Xaa36 is Pro, Xaa37 Ser, Xaa38 is Ser, Xaa39 is Gly, Xaa40 is Ala, Xaa41 is Pro, Xaa42 is Pro, Xaa43 is Pro, Xaa44 is Ser, C-terminal is amide and wherein the ε-amino group of Lys26 is substituted with a AEEA-AEEA-γ-Glu-(CH₂)ₘ—OH (AEEA=2-[2-(2-aminoethoxy)ethoxy]acetic acid) wherein m is 18.

In some embodiments, GLP-1 comprises the sequence of His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg (SEQ ID NO: 8).

In some embodiments, Extendin-4 comprises the sequence His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser (SEQ ID NO: 9).

In some embodiments, the GLP-1 analog comprises APi3709, APi3710, APi3711, APi3712, or APi3713. In certain embodiments, the GLP-1 analog comprises APi3711, APi3712, and APi3713. In certain embodiments, the GLP-1 analog comprises APi37013. In certain embodiments, the GLP-1 analog comprises APi3712.

In typical embodiments, the GLP-1 analog is resistant to enzymatic degradation. In typical embodiments, the GLP-1 analog is resistant to enzymatic degradation, including cleavage. In certain embodiments, the GLP-1 analog resistance to enzymatic degradation is determined by comparing the rate of enzymatic degradation of any one of the GLP derivates of claims 1-5 to the rate of enzymatic degradation of any one of native GLP-1 (7-36), native GLP-1(7-37), native GLP-1(7-39), semaglutide, exenatide, lyxumia, dulaglutide, tirzepatide, or liraglutide.

In some embodiments, the GLP-1 analog is resistant to a protease or a peptidase. In some embodiments, the GLP-1 analog is resistant to dipeptidyl peptidase-4 (DPP-4), or neutral endopeptidase 24.11, or a gastrointestinal enzyme.

In some embodiments, the GLP-1 analog binding affinity (IC₅₀) to the GLP-1 receptor is below 10 nM. In some embodiments, the GLP-1 analog binding affinity (IC₅₀) to the GLP-1 receptor is below 9 nM, 8 nM, 7 nM, or 6 nM. In some embodiments, the GLP-1 analog binding affinity (IC₅₀) to the GLP-1 receptor is below 5 nM. In certain embodiments, the GLP-1 analog binding affinity (IC₅₀) to GLP-1 receptor is below 4 nM, 3.5 nM, 3 nM, 2.5 nM, or 2 nM, or 1.5 nM. In certain embodiments, the GLP-1 analog binding affinity (IC₅₀) to the GLP-1 receptor is below 1 nM. In certain embodiments, the GLP-1 analog binding affinity (IC₅₀) to GLP-1 receptor is below 0.9 nM, or 0.8 nM, or 0.7 nM, or 0.6 nM, or 0.5 nM, or 0.4 nM, or 0.3 nM, or 0.2 nM, or 0.1 nM. In certain embodiments, the GLP-1 analog binding affinity (IC₅₀) to GLP-1 receptor is below 0.09 nM, or 0.08 nM, or 0.07 nM, or 0.06 nM or 0.05 nM, or 0.04 nM, or 0.03 nM, or 0.02 nM, or 0.01 nM.

In some embodiments, the peptides of the present disclosures exhibit an EC₅₀ for GLP-1 receptor activation which is in the nanomolar range. In some embodiments, the EC₅₀ of the peptide at the GLP-1 receptor is less than 1000 nM, less than 900 nM, less than 800 nM, less than 700 nM, less than 600 nM, less than 500 nM, less than 400 nM, less than 300 nM, less than 200 nM. In some embodiments, the EC₅₀ of the peptide at the GLP-1 receptor is about 100 nM or less, e.g., about 75 nM or less, about 50 nM or less, about 25 nM or less, about 10 nM or less, about 5 nM or less, or about 1 nM or less. In some or any embodiments, the peptide of the present disclosures exhibits an EC₅₀ for GLP-1 receptor activation which is in the picomolar range. In exemplary embodiments, the EC₅₀ of the GLP-1 analog peptide at the GLP-1 receptor is less than 1000 pM, less than 900 pM, less than 800 pM, less than 700 pM, less than 600 pM, less than 500 pM, less than 400 pM, less than 300 pM, less than 200 pM. In some embodiments, the EC₅₀ of the peptide at the GLP-1 receptor is about 100 pM or less, e.g., about 75 pM or less, about 50 pM or less, about 25 pM or less, about 10 pM or less, about 5 pM or less, or about 1 pM or less.

In some embodiments, the compositions of the present invention do not comprise activity at the GIP receptor.

Suitable methods of determining the EC₅₀ of a peptide for activation of a receptor, e.g., the GLP-1 receptor, are known in the art. In some embodiments, the GLP-1 analog binding affinity is measured by surface plasmon resonance or other similar methods of mass-dependent spectrometry. In some embodiments, the GLP-1 analog binding affinity is measured by a gel-shift assay. In some embodiments, the GLP-1 analog binding affinity is measured by a competition assay. In some embodiments, the GLP-1 analog binding affinity is measured by an ELISA assay.

In some embodiments, the GLP-1 analog binding is determined by imaging. In some embodiments, the GLP-1 analog binding is determined by fluorescence imaging. In some embodiments, the GLP-1 analog binding is determined by a radioligand binding assay.

In some embodiments, the GLP-1 analog binding affinity is measured using a recombinant cell line. In some embodiments, the GLP-1 analog binding affinity is measured using a mammalian recombinant cell line. In some embodiments, the GLP-1 analog binding affinity is measured using a human recombinant cell line. In some embodiments, the GLP-1 analog binding affinity is measured using a non-human recombinant cell line. In some embodiments, the recombinant cell line expresses a metabolic or hormone receptor. In some embodiments, the recombinant cell line expresses a G protein-coupled receptor. In typical embodiments, the recombinant cell line expresses a GLP-1 receptor (i.e., GLP-1R). In certain embodiments, the recombinant cell line is HEK293, HT-1080, or PER.C6.

In certain embodiments, the GLP-1 analog binding affinity (IC₅₀) to the GLP-1 receptor is determined by measuring luciferase activity using a recombinant cell reporter assay, system, or kit. In certain embodiments, the GLP-1 analog binding affinity (IC₅₀) to the GLP-1 receptor is determined by measuring luciferase activity using a recombinant cell reporter assay, wherein luciferase expression is under the control of a cAMP response element (CRE) and wherein the recombinant cell constitutively expresses GLP-1R.

### Compositions and Formulations

In certain embodiments, the GLP-1 analog exhibits an extended in vivo plasma elimination half-life of at least 5 minutes, 10 minutes, 20 minutes, 30 minutes, 40 minutes, 50 minutes, or 60 minutes. In certain embodiments, the GLP-1 analog exhibits an extended in vivo plasma elimination half-life of at least 1.5 hours, 2.0 hours, 2.5 hours, 3.5 hours, 4.0 hours, 4.5 hours, 5.0 hours, 5.5 hours, 6.0 hours, 6.5 hours, 7.0 hours, 7.5 hours, 8.0 hours, 8.5 hours, 9.0 hours, or 9.5 hours. In certain embodiments, the GLP-1 analog exhibits an extended in vivo plasma elimination half-life of at least 10 hours, at least 15 hours, or at least 20 hours. In certain embodiments, the GLP-1 analog exhibits an extended in vivo plasma elimination half-life of at least 24 hours. In certain embodiments, the GLP-1 analog exhibits an extended in vivo plasma elimination half-life of at least one week. In certain embodiments, the GLP-1 analog exhibits an extended in vivo plasma elimination half-life of at least 24 hours. In certain embodiments, the GLP-1 analog exhibits an extended in vivo plasma elimination half-life of at least 168 hours. In certain embodiments, the GLP-1 analog exhibits an extended in vivo plasma elimination half-life of at least 24 hours. In certain embodiments, the GLP-1 analog exhibits an extended in vivo plasma elimination half-life of at least one week.

In some embodiments, the composition of the present invention can include liquid, solid, or semi-solid forms. Non-limiting examples of composition forms include liquid solutions, injectable solutions, infusible solutions, dispersions, suspensions, tablets, pills, powders, liposomes, suppositories, gels, or hydrogels. In some embodiments, the composition comprises a hydrogel.

In some embodiments, the composition comprises a biocompatible polymer. Biocompatible polymers include, but are not limited to, silicone and silicone-based polymers (e.g., polydimethylsiloxane (PDMS)); liquid silicon rubber; polymethylmethacrylate (PMMA), polyurethane, styrenic block copolymers, polytetrafluoroethylene (PTFE); a natural or synthetic hydrogel; polysulfone; polyethylene; polycarbonate, polypropylene; polyamide; polyester; polymethylmethacrylate, polylactic acid (PLA), polylactide, polyglycolic acid (PGA), poly(lactic-co-glycolic acid) (PLGA), polyvinyl alcohol, any art-recognized biocompatible polymers, and any combinations thereof. In some embodiments, the biocompatible polymer comprises PLA. In some embodiments, the biocompatible polymer comprises PLGA.

In some embodiments, the biocompatible polymers comprise a molecular weight of 5,000 Da to 200,000 Da. In some embodiments, the biocompatible polymers comprise a molecular weight from 5,000 Da to 10,000 Da, from 10,000 Da to 20,000 Da, from 20,000 Da to 30,000 Da, from 30,000 Da to 40,000 Da, from 40,000 Da to 50,000 Da, from 50,000 Da to 75,000 Da, from 75,000 Da to 125,000 Da, from 125,000 Da to 150,000 Da, or from 150,000 Da to 200,000 Da. In some embodiments, the biocompatible polymer comprises a molecular weight from 10,000 Da to 18,000 Da. In some embodiments, the biocompatible polymer comprises a molecular weight from 75,000 Da to 120,000 Da.

In some embodiments, the composition of the present invention comprises a pharmaceutical solvent. In some embodiments, the pharmaceutical solvent comprises a polar aprotic solvent, a polar solvent, a nonpolar solvent, a protic solvent, or an aprotic solvent. In some embodiments, the pharmaceutical solvent comprises a polar, aprotic solvent. Polar aprotic solvents include but are not limited to, dimethylsulfoxide (DMSO), dimethylformamide (DMF), ethyl acetate, n-methyl pyrrolidone (NMP), dimethylacetamide (DMA), acetone, acetonitrile (ACN), dichloromethane, dimethylpropyleneurea, hexamethylphosphoramide, pyridine, sulfolane, tetrahydrofuran, propylene carbonate, or any art-recognized solvent. In some embodiments, the pharmaceutical solvent comprises NMP. In some embodiments, the composition further comprises a cosolvent. In some embodiments, the cosolvent comprises ACN.

In some embodiments, the composition of the present invention further comprises alcohol. Alcohols include but are not limited to, ethanol, methanol, isopropanol, or any art-recognized alcohol. In some embodiments, the alcohol comprises ethanol.

In some embodiments, the composition of the present invention comprises a microsphere, micelle, suspension, emulsion, or nanoparticle. In some embodiments, the compositions of the present invention comprise a microsphere, micelle, suspension, emulsion, or nanoparticle that contributes to the extended-release of the GLP-1 analog.

The release of the GLP-1 analog can be diffusion-controlled, chemically-controlled, biodegradable, solubility-controlled, solvent-controlled (swelling, osmosis, rupture), controlled by the extent of crosslinking and crystallinity, the size, thickness, or volume of the composition or formulation, the porosity, and the solubility of the composition or formulation (e.g. plasticizers or the additional of hydrophilic agents (e.g. glucose, mannitol)) that are rapidly dissolved and create a network or pathway for dissolution of the GLP-1 analog out of the system, or controlled by the degradation of the hydrogel scaffold. The release rate of the GLP-1 analog can also be controlled by the pH, ionic strength, temperature, magnetic field, ultrasound, or electrical stimulation. The release rate can be monomodal, bimodal, or polymodal. The release rate can include a burst phase and then a linear continuous extended-release phase.

In some embodiments, a given concentration of the GLP-1 analog remains stable following storage for several days, weeks, months, or years at a given temperature. In some embodiments, the concentration of the GLP-1 agonist that remains stable for several days, weeks, months, or years at a given temperature includes, but is not limited to, at least 10 ng/mL, 20 ng/mL, 30 ng/mL, 40 ng/mL, 50 ng/mL, 60 ng/mL, 70 ng/mL, 80 ng/mL, 90 ng/mL, 100 ng/mL, 500 ng/mL, 1000 ng/mL, 5000 ng/mL, 0.01 mg/mL, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.25 mg/mL, 1.5 mg/mL, 1.75 mg/mL, 2.0 mg/mL, 2.5 mg/mL, 3.0 mg/mL, 4.0 mg/mL, 5.0 mg/mL, 6.0 mg/mL, 7.0 mg/mL, 8.0 mg/mL, 9.0 mg/mL, or 10.0 mg/mL. In some embodiments, the GLP-1 analog remains stable for at least 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 3 months, 4 months, 5 months, 6 months, 7 months, 8 months, 9 months, 10 months, 11 months, 12 months, 2 years, 3 years, 4 years, or 5 years at a temperature that includes but is not limited to, at least −200° C., −100° C., −80° C., −70° C., −60° C., −50° C., −40° C., −30° C., −20° C., −10° C., −5° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C.

In some embodiments, the GLP-1 analog at a concentration of 1.0 mg/mL, remains stable for 1 day following storage at −80° C. In some embodiments, the GLP-1 analog at a concentration of 1.0 mg/mL, remains stable for 2 days following storage at −80° C. In some embodiments, the GLP-1 analog at a concentration of 1.0 mg/mL, remains stable for 3 days following storage at −80° C. In some embodiments, the GLP-1 analog at a concentration of 1.0 mg/mL, remains stable for 4 days following storage at −80° C. In some embodiments, the GLP-1 analog at a concentration of 1.0 mg/mL, remains stable for 5 days following storage at −80° C. In some embodiments, the GLP-1 analog at a concentration of 1.0 mg/mL, remains stable for 6 days following storage at −80° C. In some embodiments, the GLP-1 analog at a concentration of 1.0 mg/mL, remains stable for 7 days following storage at −80° C. In some embodiments, the GLP-1 analog at a concentration of 1.0 mg/mL, remains stable for 1 day following storage at 4° C. In some embodiments, the GLP-1 analog at a concentration of 1.0 mg/mL, remains stable for 2 days following storage at 4° C. In some embodiments, the GLP-1 analog at a concentration of 1.0 mg/mL, remains stable for 3 days following storage at 4° C. In some embodiments, the GLP-1 analog at a concentration of 1.0 mg/mL, remains stable for 4 days following storage at 4° C. In some embodiments, the GLP-1 analog at a concentration of 1.0 mg/mL, remains stable for 5 days following storage at 4° C. In some embodiments, the GLP-1 analog at a concentration of 1.0 mg/mL, remains stable for 6 days following storage at 4° C. In some embodiments, the GLP-1 analog at a concentration of 1.0 mg/mL, remains stable for 7 days following storage at 4° C. In some embodiments, the GLP-1 analog at a concentration of 1.0 mg/mL, remains stable for 1 day following storage at 8° C. In some embodiments, the GLP-1 analog at a concentration of 1.0 mg/mL, remains stable for 2 days following storage at 8° C. In some embodiments, the GLP-1 analog at a concentration of 1.0 mg/mL, remains stable for 3 days following storage at 8° C. In some embodiments, the GLP-1 analog at a concentration of 1.0 mg/mL, remains stable for 4 days following storage at 8° C. In some embodiments, the GLP-1 analog at a concentration of 1.0 mg/mL, remains stable for 5 days following storage at 8° C. In some embodiments, the GLP-1 analog at a concentration of 1.0 mg/mL, remains stable for 6 days following storage at 8° C. In some embodiments, the GLP-1 analog at a concentration of 1.0 mg/mL, remains stable for 7 days following storage at 8° C.

### Pharmaceutical Compositions

The disclosure herein also describes pharmaceutical compositions comprising a GLP-1 analog. The compositions of the invention are prepared by conventional processes, involving dissolving, and mixing the ingredients to give the desired composition.

In typical embodiments, the pharmaceutical composition comprises a GLP-1 analog and a pharmaceutically acceptable vehicle or carrier. In certain embodiments, the pharmaceutically acceptable carrier is a microsphere, a micelle, or a nanoparticle. In certain embodiments, the pharmaceutically acceptable carrier is a protein, such as a receptor or albumin.

In some embodiments, the pharmaceutical composition comprises any one of an isotonic agent, an excipient, a preservative, or a buffer. In some embodiments, the isotonic agent is one or more of propylene glycol, sodium chloride, potassium chloride, mannitol, sorbitol, trehalose, lactitol, xylitol, glycerol, sucrose, glycine, lactose, glucose, maltose, lysine, isoleucine, aspartic acid, L-glycine, L-histidine, arginine, myo-inositol, or polyethylene glycol.

In some embodiments, the preservative comprises any one of phenol, ra-cresol, thiomerosal, methyl paraben, propyl paraben, butyl paraben, chlorobutanol, and phenoxyethanol. In some embodiments, the buffer comprises any one of dipotassium phosphate, sodium bicarbonate, and disodium phosphate anhydrous.

In some embodiments, the pharmaceutical composition comprises a surfactant. In certain embodiments, the surfactant is at least one of polysorbate, poloxamers, ethylene/polypropylene block polymers, lecithins, alcohols, sodium lauryl sulfate, bile acids and salts thereof, polymeric surfactants, long-chain fatty acids, phospholipids, ethoxylated castor oil, polyglycolyzed glycerides, acetylated monoglycerides, sorbitan fatty acid esters, polyoxyethylene sorbitan fatty acid esters, monoglycerides, diglycerides, glycerol, glycerophospholipids, glyceroglycolipids, sphingophospholipids, sphingoglycolipids, docusate sodium, docusate calcium, docusate potassium.

In some embodiments, the pharmaceutical composition comprises a chelating agent. Suitable examples of chelating agents include ethylenediaminetetraacetic acid or its salts, and mixtures thereof.

In typical embodiments, the pharmaceutical composition remains stable after exposure to multiple free-thaw cycles. In typical embodiments, the pharmaceutical composition remains stable after exposure to an elevated temperature. Stability can be measured in any way, including visually inspecting the compositions in daylight with a dark background for any signs of turbidity, changes in color or clarity, or any other visible precipitates.

In certain embodiments, the pharmaceutical composition is at a pH range from about 7.0 to about 11.0. In certain embodiments, the pH is adjusted to about one of the following values: 7.0, 7.2, 7.3, 7.4, 7.5, 7.6. 7.7, 7.8, 7.9, 8.0.

In certain embodiments, the pharmaceutical composition pH is adjusted using a pH-adjusting agent selected from the group consisting of sodium hydroxide, potassium hydroxide, hydrochloric acid, and N-methyl glucamine.

In some embodiments, the pharmaceutical composition comprises an anti-diabetic agent or an anti-obesity agent.

### METHODS OF USE

The disclosure herein also provides for methods of treatment of human disease or disorder using a therapeutically effective amount of the GLP-1 analog. In some embodiments, the methods of treatment of human disease or disorder comprise administering the compositions, formulations, or pharmaceutical compositions described herein. In some embodiments, the human disease or disorder is a metabolic disease, cardiovascular disease, neurological disease, or inflammatory disease.

In some embodiments, the method of treating a metabolic disease comprises administering to a subject a therapeutically effective amount of a GLP-1 analog. In some embodiments, the metabolic disease comprises diabetes and/or obesity. In some embodiments the method of treating diabetes and/or obesity results in the reduction of HbA1C in the subject. In certain embodiments, the patient's glucose levels are measured by a clinician before administration of the GLP-1 analog. In some embodiments, the method of treating diabetes and/or obesity results in any one of decreasing food intake, reduction in body weight, suppression of appetite, or inducing satiety in the subject.

In some embodiments, the method of treating a cardiovascular disease comprises administering to a subject a therapeutically effective amount of a GLP-1 analog. In some embodiments, cardiovascular disease comprises heart failure, arrhythmia, congenital heart disease, atherosclerosis, peripheral artery disease, coronary artery disease, or stroke.

In some embodiments, the method of treating a neurological disease comprises administering to a subject a therapeutically effective amount of a GLP-1 analog. In some embodiments, the neurological disease comprises Alzheimer's disease, epilepsy, Parkinson's disease, autism, cerebral palsy, amyotrophic lateral sclerosis, multiple sclerosis, or Guillian-Barre syndrome.

In some embodiments, the method of treating an inflammatory disease comprises administering to a subject a therapeutically effective amount of a GLP-1 analog. In some embodiments, the inflammatory disease comprises non-alcoholic steatohepatitis (NASH), asthma, arthritis, inflammatory bowel disease, or fatty liver disease.

The disclosure herein also provides for methods of using a therapeutically effective amount of the GLP-1 analog for the treatment of human disease or disorder. In some embodiments, the methods of use comprise administering the compositions, formulations, or pharmaceutical compositions described herein. In some embodiments, the human disease or disorder is a metabolic disease, cardiovascular disease, neurological disease, or inflammatory disease. In some embodiments, the metabolic disease comprises diabetes and/or obesity. In some embodiments, cardiovascular disease comprises heart failure, arrhythmia, congenital heart disease, atherosclerosis, peripheral artery disease, coronary artery disease, or stroke. In some embodiments, the neurological disease comprises Alzheimer's disease, epilepsy, Parkinson's disease, autism, cerebral palsy, amyotrophic lateral sclerosis, multiple sclerosis, or Guillian-Barre syndrome. In some embodiments, the inflammatory disease comprises non-alcoholic steatohepatitis (NASH), asthma, arthritis, inflammatory bowel disease, or fatty liver disease.

In some embodiments, the GLP-1 analog is administered by oral, intravenous, parenteral, transdermal, intramuscular, rectal, vaginal, or topical administration. In some embodiments, the GLP-1 analog is delivered by a transdermal patch. In some embodiments, the GLP-1 analog is delivered by an implant device or method.

In some embodiments, the GLP-1 analog is administered by injection. In some embodiments, the GLP-1 analog is administered by injection by a syringe. In some embodiments, the GLP-1 analog is administered orally by tablet, pill, capsule, elixir, syrup, extract, or solution. In some embodiments, the GLP-1 analog is administered by injection at an interval of twice weekly. In some embodiments, the GLP-1 analog is administered by injection at an interval of once weekly. In some embodiments, the GLP-1 analog is administered by injection at an interval of twice monthly. In some embodiments, the GLP-1 analog is administered by injection at an interval of once monthly. In some embodiments, the GLP-1 analog is administered by injection at an interval of once every other month. In some embodiments, the GLP-1 analog is administered by injection at an interval of twice yearly. In some embodiments, the GLP-1 analog is administered by injection at an interval of once yearly.

In some embodiments, the compositions, formulations, or pharmaceutical compositions disclosed herein are useful in the field of medicine for the treatment, prophylaxis, palliation, or amelioration of metabolic, cardiovascular, neurological, or inflammatory diseases and/or disorders.

The present disclosure provides a method for administering to a subject in need thereof, including a human subject, the compositions, formulations, or pharmaceutical compositions disclosed herein to slow, stop, or reverse disease progression. As a nonlimiting example, disease progression can be measured by tests or diagnostic tool(s) known to those skilled in the art. As another non-limiting example, disease progression can be measured by changes in the pathological features of the pancreas, brain, or other tissues of the subject.

In some embodiments, a therapeutically effective amount of the GLP-1 analog is administered to a subject. In some embodiments, the effective amount to be employed therapeutically will depend, for example, upon the therapeutic context and objectives. One skilled in the art will appreciate that the appropriate dosage levels for treatment, according to some embodiments, will thus vary depending, in part, upon the molecule delivered, the indication for which the GLP-1 analog is being used, the route of administration, and the size (body weight, body surface or organ size) and/or condition (the age and general health) of the subject. In some embodiments, the clinician can titer the dosage and modify the route of administration to obtain the optimal therapeutic effect.

In some embodiments, a therapeutically effective amount of compositions, formulations, or pharmaceutical compositions disclosed herein inhibits and/or prevents a particular disorder, and/or the symptoms of the disorder.

In some embodiments, the GLP-1 analog and the compositions, formulations, or pharmaceutical compositions disclosed herein comprising the GLP-1 analogs are delivered systemically. For example, in some embodiments, the GLP-1 analogs are administered through intravenous injection. In some embodiments, the GLP-1 analogs are administered through subcutaneous injection. In some embodiments, GLP-1 analogs are administered through intramuscular injection. In some embodiments, the GLP-1 analogs are administered through intravenous infusion. In some embodiments, the systemically delivered GLP-1 analogs are capable of crossing the blood-brain barrier.

### Kits and Methods of Manufacture

In some embodiments, methods of making the GLP-1 analogs and the compositions, formulations, or pharmaceutical compositions disclosed herein are envisioned.

The GLP-1 analog and the compositions, formulations, or pharmaceutical compositions disclosed herein can be designed and/or synthesized using any suitable method known in the art.

In some embodiments, the disclosure provides a kit comprising a GLP-1 analog and a composition, formulation, or pharmaceutical composition disclosed herein comprising the GLP-1 analog. In some embodiments, a kit includes a GLP-1 analog and a composition, formulation, or pharmaceutical composition disclosed herein comprising the GLP-1 analog, and instructions for use. In some embodiments, the kits comprise, in a suitable container, a GLP-1 analog and a composition, formulation, or pharmaceutical composition disclosed herein comprising the GLP-1 analog, one or more controls, and various buffers, reagents, enzymes, and other standard ingredients known in the art.

The container can include at least one vial, well, test tube, flask, bottle, syringe, or other container means, into which a GLP-1 analog and a composition, formulation, or pharmaceutical composition disclosed herein comprising the GLP-1 analog can be placed, and in some instances, suitably aliquoted. Where an additional component is provided, the kit can contain additional containers into which this component can be placed. The kits can also include a means for containing a GLP-1 analog and any other reagent containers in close confinement for commercial sale. Such containers can include injection or blow-molded plastic containers into which the desired vials are retained. Containers and/or kits can include labeling with instructions for use and/or warnings.

In some embodiments, a kit comprises a container comprising a GLP-1 analog and a pharmaceutically acceptable carrier, or a pharmaceutical composition comprising the GLP-1 analog, and instructions for treating or delaying the progression of a metabolic disease, cardiovascular disease, neurological disease, or inflammatory disease in a subject in need thereof. In some embodiments, a kit comprises a container comprising a GLP-1 analog and a pharmaceutically acceptable carrier, or a pharmaceutical composition comprising the GLP-1 analog, and instructions for administering the GLP-1 analog to a subject in need thereof, alone or in combination with another agent, for treating or delaying progression of a metabolic disease, cardiovascular disease, neurological disease, or inflammatory disease in the subject.

### Definitions

In this disclosure, “comprises,” “comprising,” “containing,” “having,” and the like can have the meaning ascribed to them in U.S. patent law and can mean “includes,” “including,” and the like; the terms “consisting essentially of” or “consists essentially” likewise have the meaning ascribed in U.S. patent law and these terms are open-ended, allowing for the presence of more than that which is recited so long as basic or novel characteristics of that which is recited are not changed by the presence of more than that which is recited, but excludes prior art embodiments.

Unless specifically stated or obvious from context, as used herein, the terms “a,” “an,” and “the” are understood to be singular or plural.

Unless specifically stated or obvious from context, as used herein, the term “or” is understood to be inclusive.

As used herein, the term “about,” unless indicated otherwise, refers to the recited value, e.g., amount, dose, temperature, time, percentage, etc., ±10%, ±9%, ±8%, ±7%, ±6%, ±5%, 4%, ±3%, ±2%, or ±1%.

As used herein, the term “subject” herein to refers to any mammal, including humans, domestic and farm animals, and zoo, sports, and pet animals, such as dogs, horses, cats, and agricultural use animals including cattle, sheep, pigs, and goats. One preferred mammal is a human, including adults, children, and the elderly. A subject may also be a pet animal, including dogs, cats, and horses. Preferred agricultural animals would be pigs, cattle, and goats. A “patient” is a human subject.

The phrases “therapeutically effective amount” and “effective amount” and the like, as used herein, indicate an amount necessary to administer to a patient, or to a cell, tissue, or organ of a patient, to achieve a therapeutic effect, such as an ameliorating or alternatively a curative effect. The effective amount is sufficient to elicit the biological or medical response of a cell, tissue, system, animal, or human that is being sought by a researcher, veterinarian, medical doctor, or clinician. Determination of the appropriate effective amount or therapeutically effective amount is within the routine level of skill in the art.

The terms “administering”, “administer”, “administration” and the like, as used herein, refer to any mode of transferring, delivering, introducing, or transporting a therapeutic agent to a subject in need of treatment with such an agent. Such modes include but are not limited to, intraocular, oral, topical, intravenous, intraperitoneal, intramuscular, intradermal, intranasal, and subcutaneous administration.

As used herein, the terms “GLP-1 analog” or “GLP-1 receptor agonist” or “GLP-1 agonist” are used interchangeably and refer to a peptide that activates the GLP-1 receptor.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations can be used, e.g., bp, base pair(s); kb, kilobase(s); pl, picoliter(s); s or sec, second(s); min, minute(s); h or hr, hour(s); aa, amino acid(s); nt, nucleotide(s); and the like.

The practice of the present invention will employ, unless otherwise indicated, conventional methods of protein chemistry, biochemistry, recombinant DNA techniques, and pharmacology, within the skill of the art.

### Example 1: Binding Affinity of GLP-1 Analogs to GLP-1R

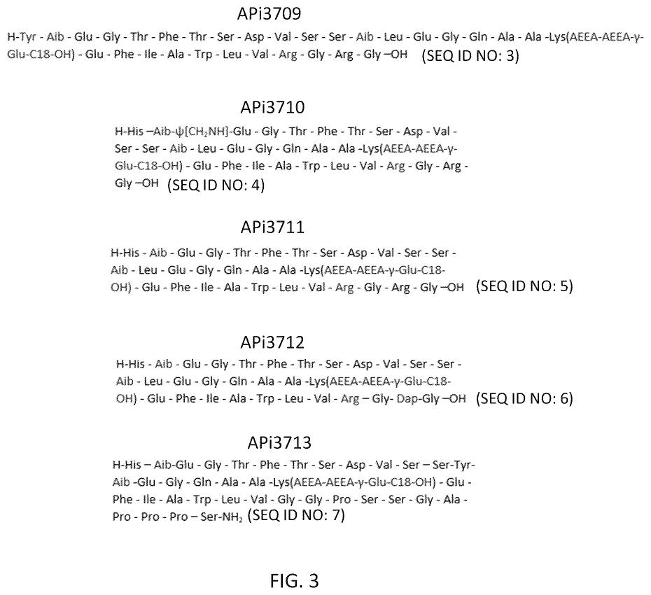

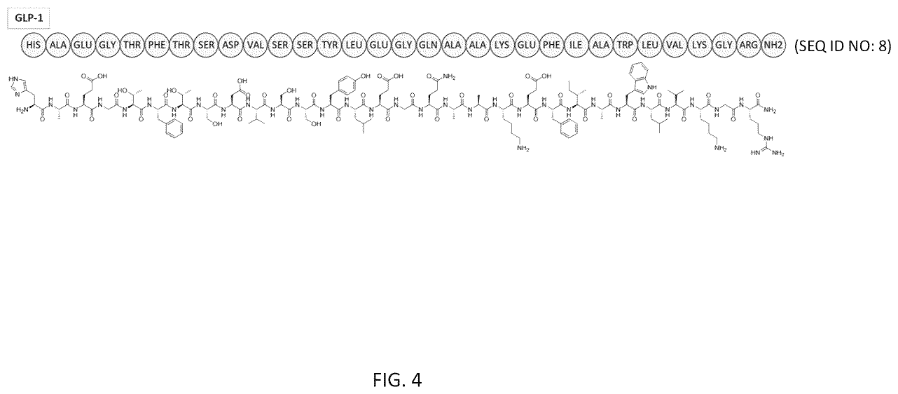

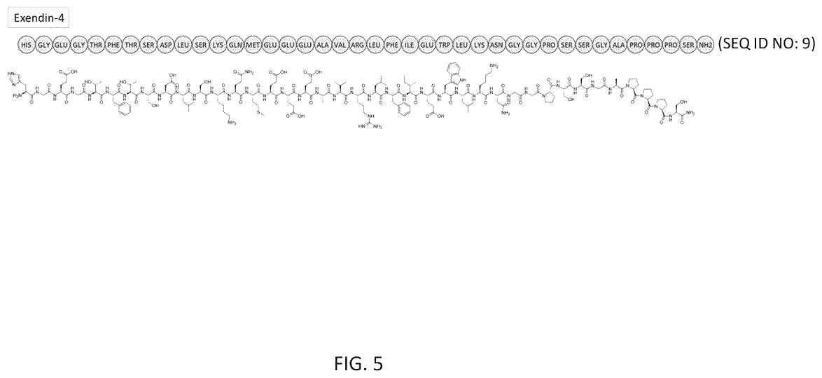

GLP-1 analogs APi3709, APi3710, APi3711, APi3712, and APi3713 were synthesized and purified using standard methods (see Table 1). The sequences are described herein and provided in FIG. 3. For reference, the sequence of native GLP-1(7-36) is provided in FIG. 4 and the exendin-4 sequence is provided in FIG. 5.

TABLE 1
GLP-1 Analog Characteristics

| | Mol. | Purity |
|---|---|---|
| Analog | Weight | (%) |
| APi3709 | 4059.1 | 98.92 |
| APi3710 | 4019.1 | 97.07 |
| APi3711 | 4033.1 | 99.37 |
| APi3712 | 3963.1 | 96.03 |
| APi3713 | 4547.3 | 97.34 |

### CRE-Luciferase Reporter Assay

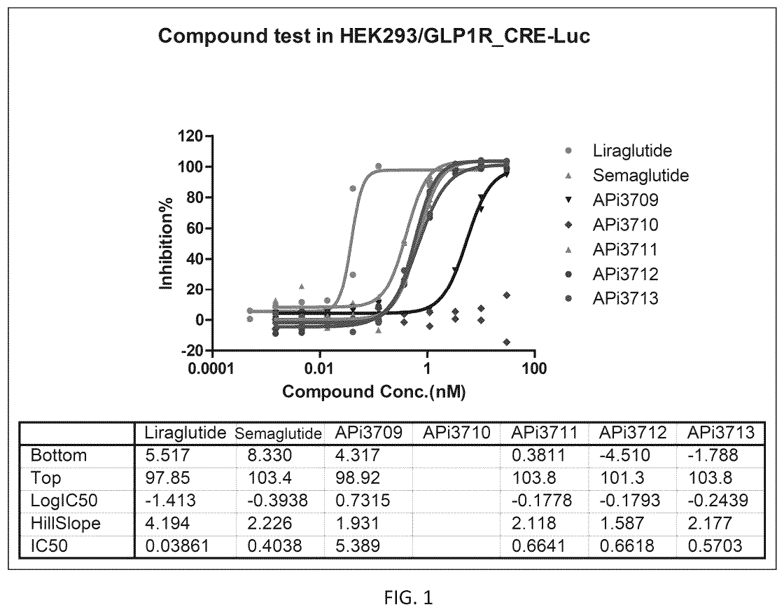

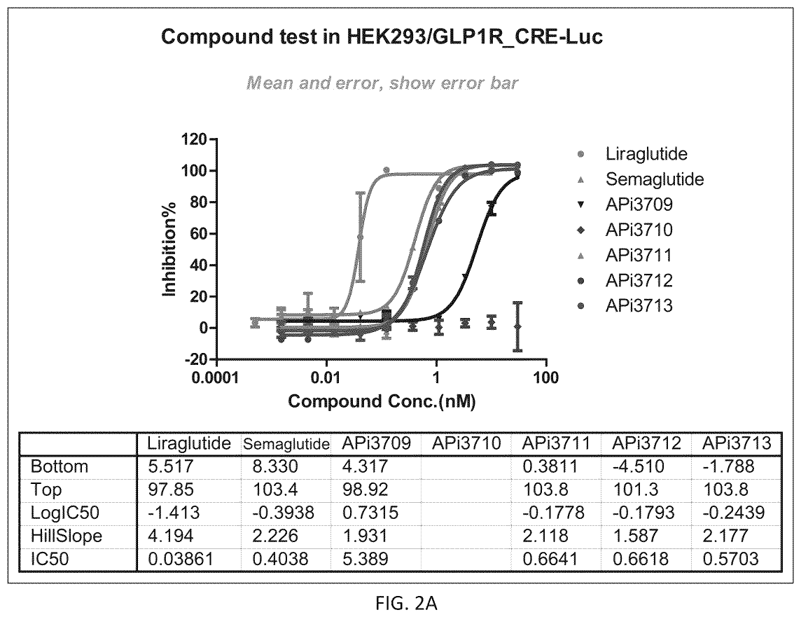

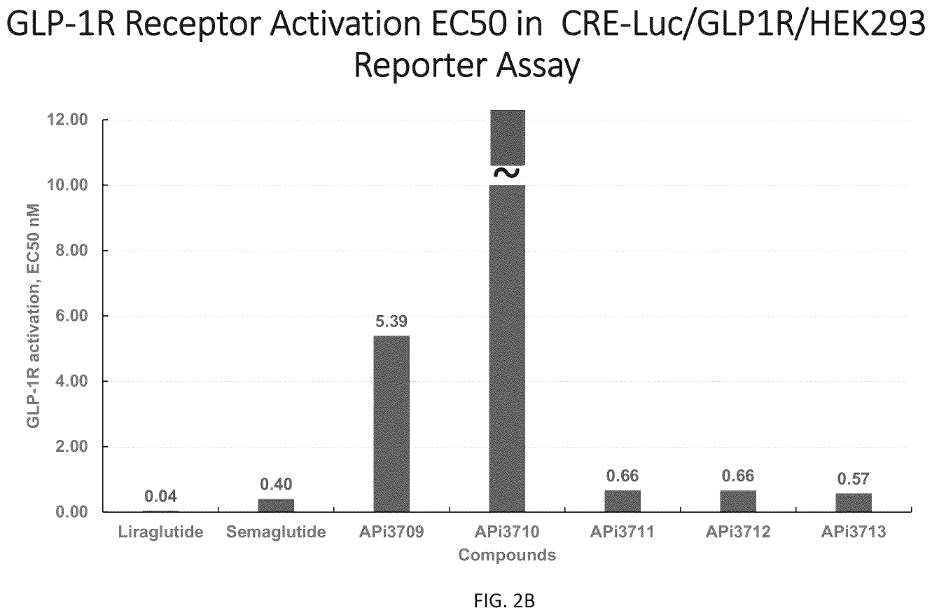

The binding affinity of the analogs to GLP-1-receptor (GLP-1R) was determined using recombinant HEK293 cells expressing the firefly luciferase gene under the control of cAMP response element (CRE) and with constitutive expression of human GLP-1R. Activation of GLP-1R in these cells by GLP-1 analog binding can be monitored by measuring luciferase activity. The GLP-1 analog binding affinities were compared to the binding affinities of liraglutide and semaglutide. The results of the binding assay are shown in FIG. 1 and FIGS. 2A-2B (FIG. 2A has the error bars included).

The biological IC₅₀ of APi3709 (˜5.4 nM) is significantly different than the IC₅₀ of liraglutide (˜0.03 nM) and semaglutide (˜0.4). APi3709 has a five amino acid residue difference from GLP-1 (7-36). Two of the five changes are different from that of semaglutide, which changed the N-terminal His1 to Tyr1 and Tyr13 to Aib13.

The biological IC₅₀ of APi3710 is significantly different from the IC₅₀ of liraglutide (˜0.03 nM) and semaglutide (˜0.4). APi3710 has a four amino acid residue difference from GLP-1 (7-36). Two of the four changes are different from that of semaglutide, which changed the N-terminal Ala2 to Aib-ψ[CH₂NH] and Tyr13 to Aib13.

The biological IC₅₀ of APi3711 (˜0.66 nM) is similar the IC₅₀ of liraglutide (˜0.03 nM) and semaglutide (˜0.4). APi3711 has a four amino acid residue difference from GLP-1 (7-36). One of the four changes is different from that of semaglutide, which changed the Tyr13 to Aib13.

The biological IC₅₀ of APi3712 (˜0.66 nM) is similar the IC₅₀ of liraglutide (˜0.03 nM) and semaglutide (˜0.4). APi3712 has a four amino acid residue difference from GLP-1 (7-36). Two of the four changes are different from that of semaglutide, which changed the Tyr13 to Aib13 and additional Dap (L-2,3-diaminopropionic acid).

The biological IC₅₀ of APi3713 (˜0.57 nM) is similar the IC₅₀ of liraglutide (˜0.03 nM) and semaglutide (˜0.4). APi3713 has multiple amino acid residue differences than GLP-1 (7-36). Multiple amino acids are different from that of semaglutide, which changed the Tyr13 to Aib13 an additional 11 amino acid residues that are the same as the C-terminal of Exenadin-4.

### Cell-Based Tag-Lite® GLP-1R Receptor Binding FRET Assay

The Tag-Lite® Glucagon GLP-1 Labeled Cells Express GLP-1R. The GLP-1R is Covalently labeled with a terbium compound which functions as a fluorescent marker (tag-lite). When the GLP-1R receptor ligand is labeled with another fluorescent marker (“red agonist”) and it binds to the GLP-1R, the two fluorescent markers (the tag-lite and the red agonist) are in close proximity to each other. Due to the proximity, the fluorescent energy emitted by the tag-lite is transferred to the fluorescent marker of the GLP-1 red agonist ligand (fluorescence resonance energy transfer or FRET).

When the tag-lite on the GLP-1 receptor is exposed to 340 nm of excitation light, it produces 616 nm of emitted light. Extendin-4 or any other GLP-1 receptor agonists (positive control) can compete with the red agonist for the GLP-1R binding site. The replacement of the red agonist in the binding of the GLP-1R by a different ligand disrupts the fluorescence transfer resulting in a decrease of 616 nm light emission.

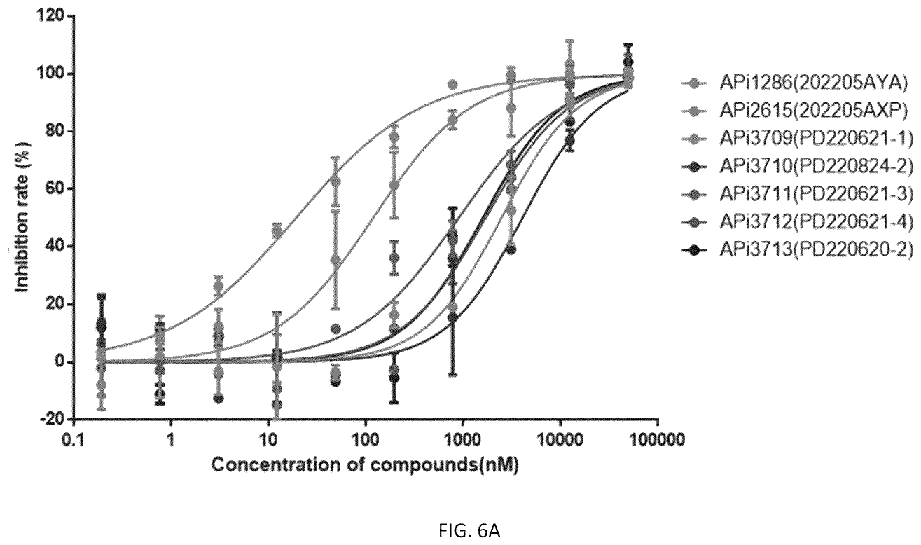

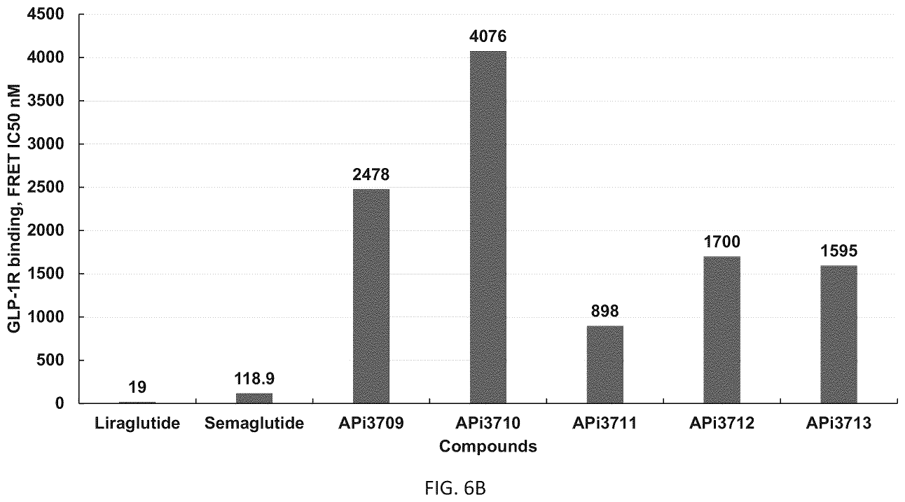

GLP-1 analogs APi3709, APi3710, APi3711, APi3712, and APi3713 showed dose dependent binding to the GLP-1 receptor compared to liraglutide and semaglutide (positive controls). The most active GLP-1 analogs were APi3711, APi3712, and APi3713, as seen in Table 2 and FIGS. 6A-6B.

TABLE 2
GLP-1 analog IC50 Measurements from Tag-Lite GLP-1R
Binding FRET Assay in the Absence of HSA

| | Liraglutide | Semaglutide | APi3709 | APi3710 | APi3711 | APi3712 | APi3713 |
|---|---|---|---|---|---|---|---|
| IC50 (nM) | 19 | 118.9 | 2478 | 4076 | 898 | 1700 | 1595 |

Conjugation with a fatty acid, a natural human serum albumin (HSA) ligand, is a technique to extend the serum half-life of therapeutic peptides in vivo by taking advantage of the long serum half-life of HSA. 2% HSA concentration mimics the HSA concentration in human blood.

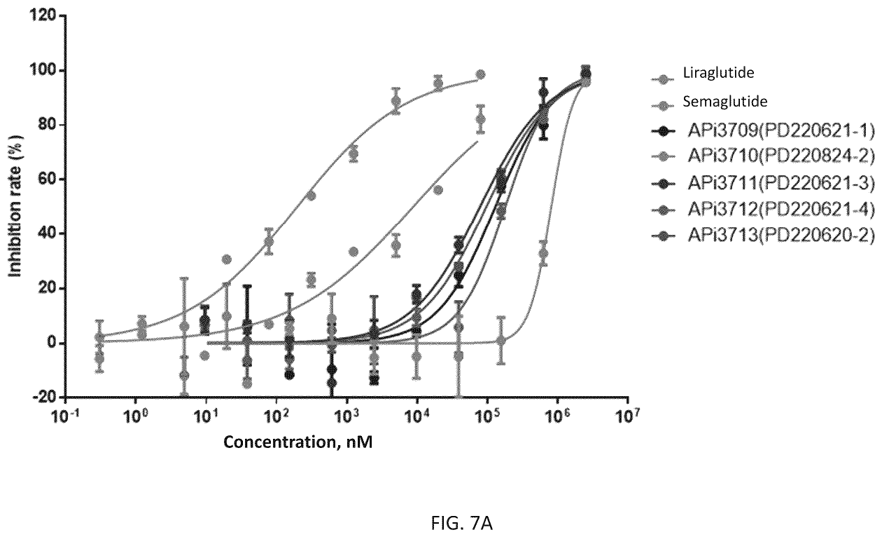

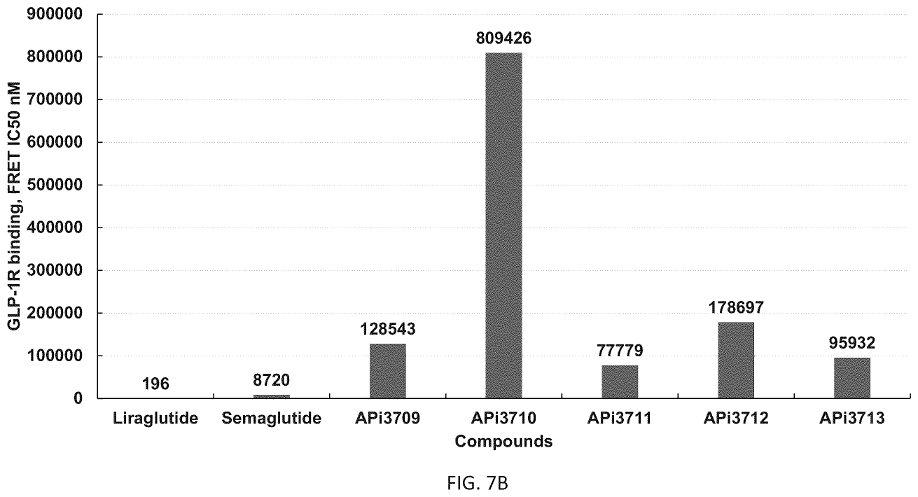

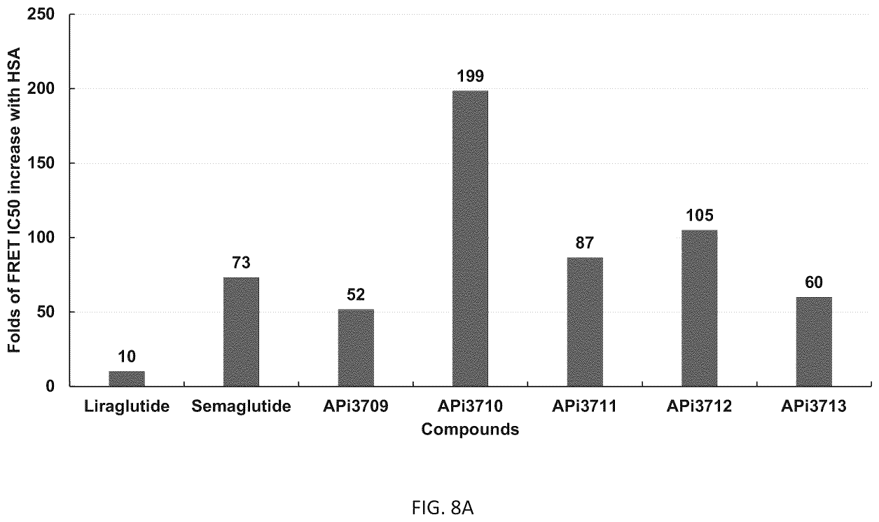

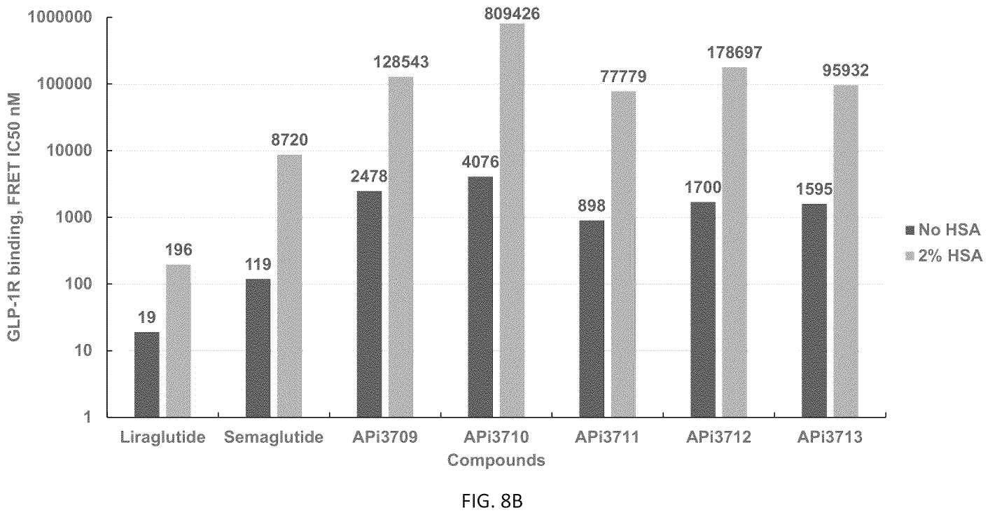

GLP-1 analogs APi3709, APi3710, APi3711, APi3712, and APi3713 showed dose dependent binding to the GLP-1 receptor in the presence of 2% HSA, as compared to liraglutide and semaglutide, however, the activity of all compounds tests was lower in the presence of 2% HSA, as shown in Table 3 and FIGS. 7A-7B and FIGS. 8A-8B.

TABLE 3
GLP-1 analog IC50 Measurements from Tag-Lite
GLP-1R Binding FRET Assay with 2% HAS

| | Liraglutide | Semaglutide | APi3709 | APi3710 | APi3711 | APi3712 | APi3713 |
|---|---|---|---|---|---|---|---|
| IC50 (nM) | 196 | 8720 | 128543 | 809426 | 77779 | 178697 | 95932 |

### Example 2: GLP-1 and Glucagon Receptor Modulator's Cross Reactivity

Glucagon and GLP-1 share ˜50% amino acid sequence identity. Similarly, glucagon receptor and GLP-1R share ˜50% amino acid sequence identity. The cross reactivity of GLP-1R, GIPR, and glucagon receptor (GCGR) modulators can have complex and interconnected biological effects. GCGR stimulation leads to the activation of adenylate cyclase and increased levels of intracellular cAMP, as well as increased levels of intracellular Ca²⁺.

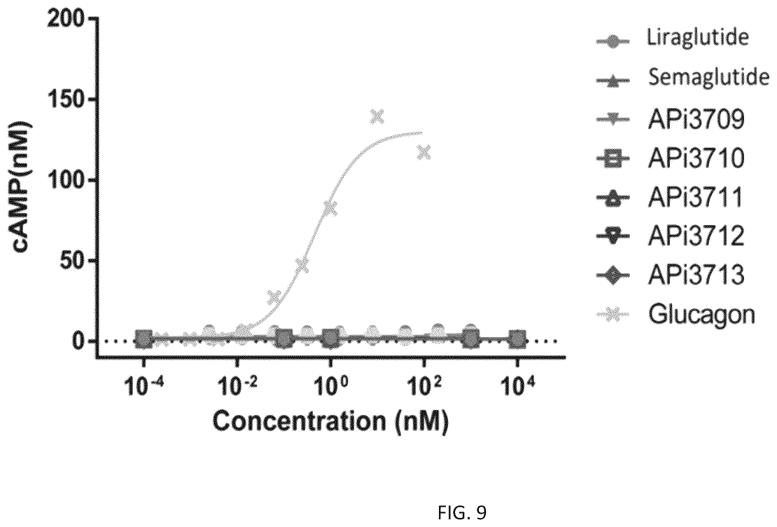

The effect of GLP-1 analogs APi3709, APi3710, APi3711, APi3712, and APi3713 on cAMP production in HEK293/GCGR/Gα15 cells was tested in a dose-dependent format using glucagon as a positive control, as shown in FIG. 9.

cAMP concentration in cell lysates was measured using an HTRF cAMP assay. cAMP produced intracellularly was detected via competition between intracellular cAMP and d2-labeled cAMP. An increase in intracellular cAMP leads to disruption of the FRET signal, whereas a decrease in intracellular cAMP results in a higher FRET signal. None of the GLP-1 analogs APi3709, APi3710, APi3711, APi3712, and APi3713 showed GCGR activation in the HEK293/GCGR/Gα15 cell-based assay (FIG. 9).

### Example 3: Structure-Activity Relationship

The structure-activity relationship of GLP-1 analogs was determined using standard methods and the conclusions are as indicated in Table 4.

TABLE 4
Structure-Activity Relationship of GLP-1 analogs

| Structure | Activity |
|---|---|
| Reduced peptide bond Aib-ψ[CH2NH]-Xaa | Substantially reduces activity |
| His→Tyr | Reduces activity |
| Arg→Dap | Slightly reduces activity |
| Tyr→Aib | Slightly reduces activity |
| Arg-Gly-Arg-Gly (SEQ ID NO: 10)→Gly- | Neutral to slightly reduces activity |
| Gly-Pro-Ser-Ser-Gly-Ala-Pro-Pro-Pro-Ser- | |
| NH2 (SEQ ID NO: 11) | |
| Ala→Aib, C16→C18 | Reduces activity |

### Example 4: GLP-1 Analog Adhesion to Plastic

The adhesion of GLP-1 analogs APi3709, APi3710, APi3711, APi3712, and APi3713 to plastic and glass was determined using standard methods. The GLP-1 analogs were prepared in a solution of PBS at a concentration slightly higher (˜2-3 fold) than the lower limit of quantification of the HPLC method. The GLP-1 analog-PBS solution was transferred between 5 polypropylene tubes and the intact solution of peptide was analyzed by HPLC after transfer (Table 5). Compared to semaglutide, the GLP-1 analogs APi3709, APi3710, APi3711, APi3712, and APi3713 each showed less adhesion to plastic.

TABLE 5
GLP-1 Analog Adhesion to Plastic

| | | | | | | Area Ratio |
|---|---|---|---|---|---|---|
| | Concen- | | | | | (Initial/ |
| GLP-1 | tration | | | | Area | Final |
| analog | (μg/mL) | HPLC Peak Areas | | | (Mean) | Tube, %) |
| Semaglutide | 1.6 | Initial | Injection 1 | 18.8 | 19.4 | 80 |
| | | Tube | Injection 2 | 20.0 | | |
| | | Final | Injection 1 | 16.8 | 15.6 | |
| | | Tube | Injection 2 | 14.3 | | |
| APi3709 | 1.6 | Initial | Injection 1 | 21.7 | 22.3 | 86 |
| | | Tube | Injection 2 | 22.8 | | |
| | | Final | Injection 1 | 20.9 | 19.1 | |
| | | Tube | Injection 2 | 17.3 | | |
| APi3710 | 1.6 | Initial | Injection 1 | 14.0 | 15.2 | 93 |
| | | Tube | Injection 2 | 16.4 | | |
| | | Final | Injection 1 | 12.7 | 14.2 | |
| | | Tube | Injection 2 | 15.7 | | |
| APi3711 | 1.6 | Initial | Injection 1 | 19.5 | 20.0 | 91 |
| | | Tube | Injection 2 | 20.5 | | |
| | | Final | Injection 1 | 17.0 | 18.2 | |
| | | Tube | Injection 2 | 19.3 | | |
| APi3712 | 1.6 | Initial | Injection 1 | 21.3 | 21.9 | 94 |
| | | Tube | Injection 2 | 22.5 | | |
| | | Final | Injection 1 | 19.8 | 20.5 | |
| | | Tube | Injection 2 | 21.1 | | |
| APi3713 | 1.6 | Initial | Injection 1 | 28.6 | 29.3 | 90 |
| | | Tube | Injection 2 | 30.0 | | |
| | | Final | Injection 1 | 27.0 | 26.5 | |
| | | Tube | Injection 2 | 26.0 | | |

### Example 4: GLP-1 Analog Stability in Formulation

The stability of GLP-1 analogs APi3709, APi3710, APi3711, APi3712, and APi3713 in formulation was determined using standard methods. The GLP-1 analogs were prepared in a solution of PBS at a concentration of 0.5-1 mg/mL. The GLP-1 analog-PBS solution was aliquoted in 250 uL aliquots in polypropylene tubes and placed at 4° C. or −80° C. The purity and concentration of the stored solutions was determined by HPLC after 1, 3, and 7 days of incubation at 4° C. or −80° C.

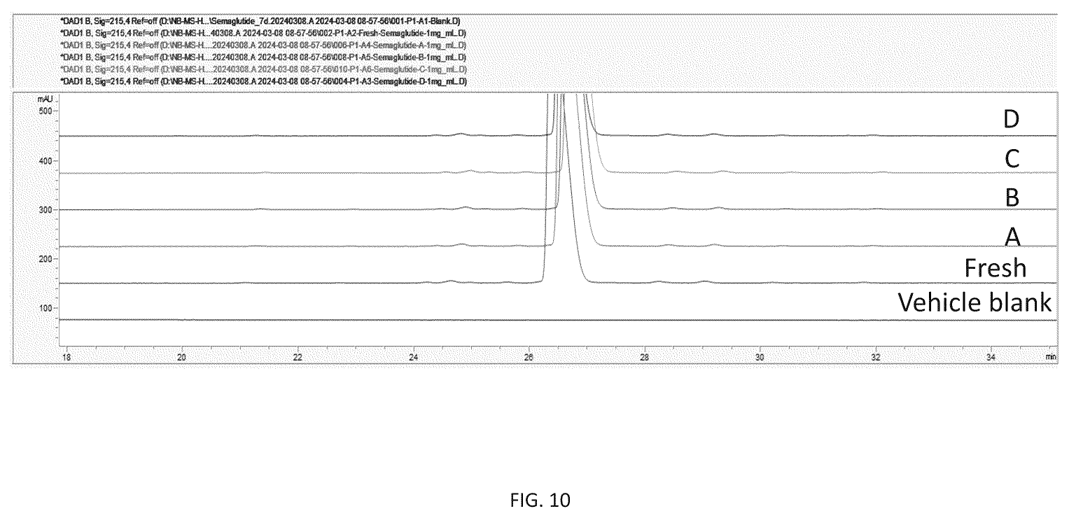

The stability of semaglutide is shown in Table 6 and FIG. 10. No significant change of purity was observed after semaglutide storage at 4° C. for 7 days. The concentration of the formulation remained consistent at 1 mg/mL.

TABLE 6
Semaglutide Stability in Formulation

| | | | | HPLC |
|---|---|---|---|---|
| | | Concentration | | Purity |
| Compound | Formulation | (mg/mL) | | (%) |
| Semaglutide | Fresh | Injection 1 | 1.00 | 98.18 |
| | (Actual = 1.00 mg/mL) | Injection 2 | 1.00 | 98.17 |
| | A | Injection 1 | 1.00 | 98.17 |
| | −80° C.-7 d | Injection 2 | 1.00 | 98.22 |
| | B | Injection 1 | 1.00 | 98.17 |
| | 4° C.-1 d | Injection 2 | 1.00 | 98.17 |
| | C | Injection 1 | 1.05 | 98.19 |
| | 4° C.-3 d | Injection 2 | 1.05 | 98.18 |
| | D | Injection 1 | 1.00 | 98.17 |
| | 4° C.-7 d | Injection 2 | 1.00 | 98.19 |

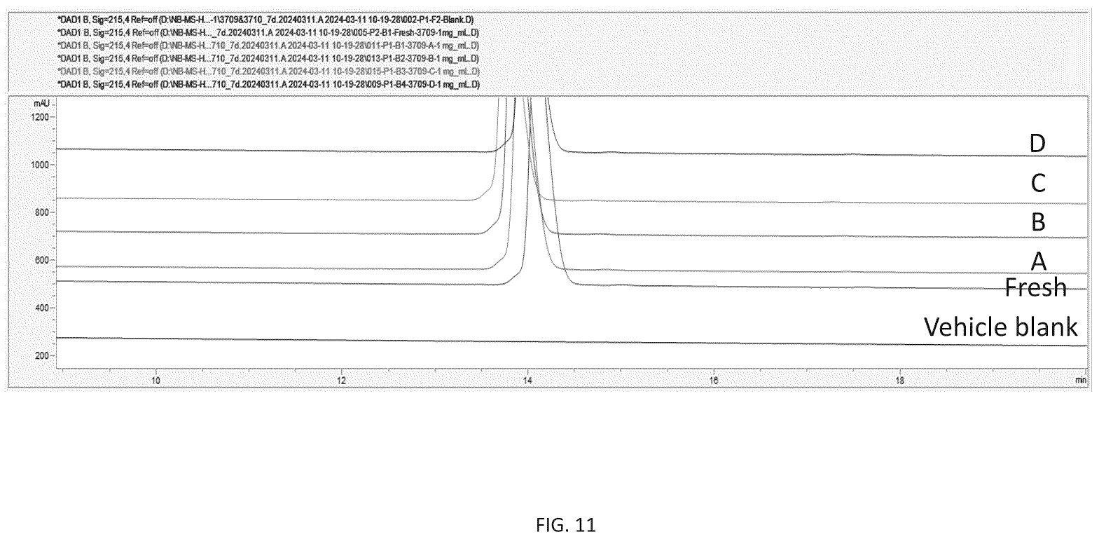

The stability of APi3709 is as shown in Table 7 and FIG. 11. No significant change of purity was observed after APi3709 storage at 4° C. or −80° C. for 7 days. The concentration of the formulation remained consistent at 1 mg/mL.

TABLE 7
APi3709 Stability in Formulation

| | | | | HPLC |
|---|---|---|---|---|
| | | Concentration | | Purity |
| Compound | Formulation | (mg/mL) | | (%) |
| APi3709 | Fresh | Injection 1 | 0.98 | 99.57 |
| | (Actual = 1.00 mg/mL) | Injection 2 | 0.98 | 99.55 |
| | A | Injection 1 | 0.96 | 99.56 |
| | −80° C.-7 d | Injection 2 | 0.97 | 99.54 |
| | B | Injection 1 | 1.03 | 99.55 |
| | 4° C.-1 d | Injection 2 | 1.03 | 99.55 |
| | C | Injection 1 | 0.98 | 99.54 |
| | 4° C.-3 d | Injection 2 | 0.97 | 99.56 |
| | D | Injection 1 | 1.02 | 99.57 |
| | 4° C.-7 d | Injection 2 | 1.02 | 99.58 |

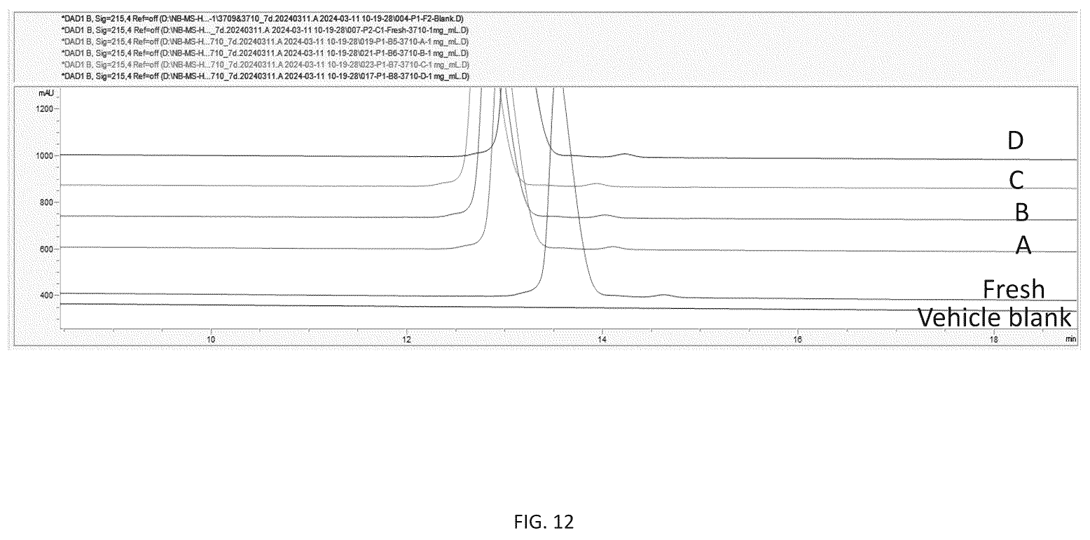

The stability of APi3710 is shown in Tables 8 and 9 and FIG. 12. Slight degradation of purity was observed after APi3710 storage at 4° C. or −80° C. for 7 days.

TABLE 8
APi3710 Stability in Formulation

| | | | | HPLC |
|---|---|---|---|---|
| | | Concentration | | Purity |
| Compound | Formulation | (mg/mL) | | (%) |
| APi3710 | Fresh | Injection 1 | 0.97 | 99.24 |
| | (Actual = 0.98 mg/mL) | Injection 2 | 0.97 | 99.23 |
| | A | Injection 1 | 0.96 | 99.13 |
| | −80° C.-7 d | Injection 2 | 0.94 | 99.15 |
| | B | Injection 1 | 0.99 | 99.12 |
| | 4° C.-1 d | Injection 2 | 0.99 | 99.14 |
| | C | Injection 1 | 0.98 | 99.05 |
| | 4° C.-3 d | Injection 2 | 0.99 | 99.04 |
| | D | Injection 1 | 0.99 | 99.00 |
| | 4° C.-7 d | Injection 2 | 0.99 | 99.00 |

TABLE 9
Impurity in APi3710

| | | HPLC Purity | |
|---|---|---|---|
| | | (area %) | |
| | Retention Time (min) | 13.56 | 14.62 |
| Formulation | RRT | 1.00 | 1.08 |
| Fresh | Injection 1 | 99.12 | 0.88 |
| | Injection 2 | 99.09 | 0.91 |
| A | Injection 1 | 98.87 | 1.13 |
| −80° C.-7 d | Injection 2 | 98.84 | 1.16 |
| B | Injection 1 | 98.80 | 1.20 |
| 4° C.-1 d | Injection 2 | 98.85 | 1.15 |
| C | Injection 1 | 98.77 | 1.23 |
| 4° C.-3 d | Injection 2 | 98.77 | 1.23 |
| D | Injection 1 | 98.75 | 1.25 |
| 4° C.-7 d | Injection 2 | 98.75 | 1.25 |

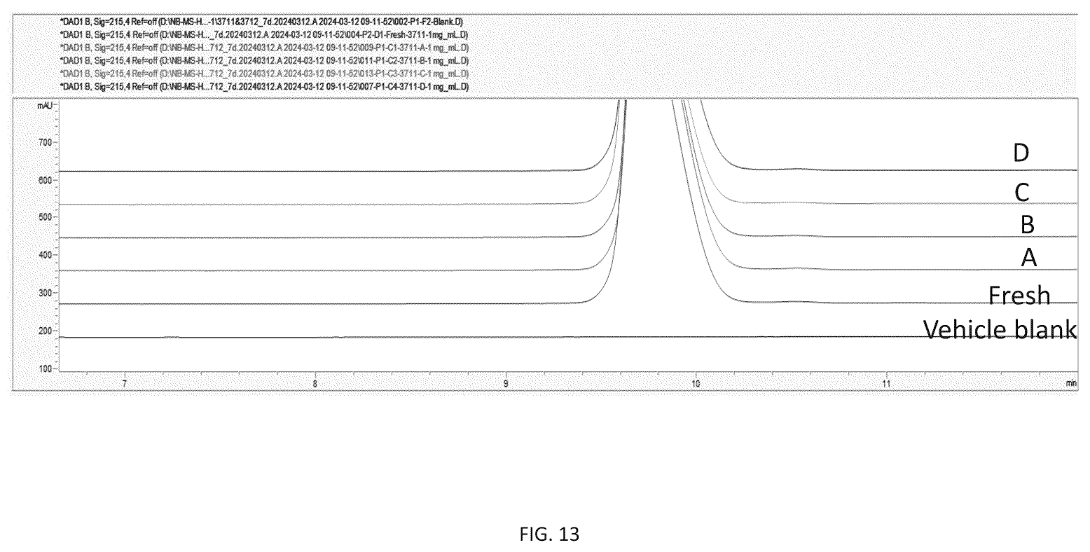

The stability of APi3711 is shown in Table 10 and FIG. 13. No significant change of purity was observed after APi3711 storage at 4° C. or −80° C. for 7 days. The concentration of the formulation remained consistent at 1 mg/mL.

TABLE 10
APi3711 Stability in Formulation

| | | Concentration | | HPLC |
|---|---|---|---|---|
| | | of HPLC | | Purity |
| Compound | Formulation | (mg/mL) | | (%) |
| APi3711 | Fresh | Injection 1 | 1.00 | 99.63 |
| | (Actual = 1.00 mg/mL) | Injection 2 | 1.00 | 99.62 |
| | A | Injection 1 | 1.00 | 99.62 |
| | −80° C.-7 d | Injection 2 | 1.00 | 99.62 |
| | B | Injection 1 | 1.00 | 99.61 |
| | 4° C-.1 d | Injection 2 | 1.00 | 99.64 |
| | C | Injection 1 | 0.89 | 99.62 |
| | 4° C.-3 d | Injection 2 | 0.89 | 99.63 |
| | D | Injection 1 | 1.00 | 99.64 |
| | 4° C.-7 d | Injection 2 | 1.00 | 99.64 |

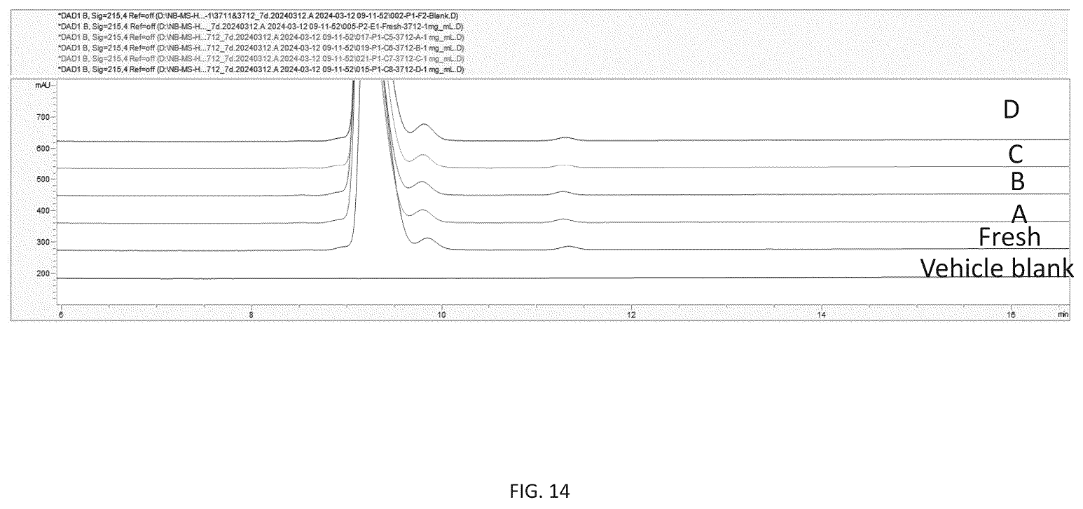

The stability of APi3712 is shown in Tables 11 and 12 and FIG. 14. Slight degradation of purity was observed after APi3712 storage at 4° C. or −80° C. for 7 days.

TABLE 11
APi3712 Stability in Formulation

| | | Concentration | | HPLC |
|---|---|---|---|---|
| | | of HPLC | | Purity |
| Compound | Formulation | (mg/mL) | | (%) |
| APi3712 | Fresh | Injection 1 | 0.98 | 95.97 |
| | (Actual = 0.99 mg/mL) | Injection 2 | 0.98 | 95.96 |
| | A | Injection 1 | 0.97 | 95.70 |
| | −80° C.-7 d | Injection 2 | 0.97 | 95.66 |
| | B | Injection 1 | 0.98 | 95.55 |
| | 4° C.-1 d | Injection 2 | 0.98 | 95.51 |
| | C | Injection 1 | 0.92 | 95.23 |
| | 4° C.-3 d | Injection 2 | 0.92 | 95.22 |
| | D | Injection 1 | 0.96 | 94.75 |
| | 4° C.-7 d | Injection 2 | 0.96 | 94.73 |

TABLE 12
Impurity in APi3712

| | | Purity (area %) | | | |
|---|---|---|---|---|---|
| | Time (min) | 8.59 | 9.22 | 9.86 | 11.34 |
| Formulation | RRT | 0.93 | 1.00 | 1.07 | 1.23 |
| Fresh | Injection 1 | 0.14 | 95.99 | 3.10 | 0.78 |
| | Injection 2 | 0.14 | 95.96 | 3.12 | 0.79 |
| A | Injection 1 | 0.13 | 95.71 | 3.38 | 0.78 |
| −80° C.-7 d | Injection 2 | 0.13 | 95.67 | 3.43 | 0.78 |
| B | Injection 1 | 0.14 | 95.54 | 3.53 | 0.79 |
| 4° C.-1 d | Injection 2 | 0.14 | 95.53 | 3.55 | 0.79 |
| C | Injection 1 | 0.12 | 95.33 | 3.80 | 0.75 |
| 4° C.-3 d | Injection 2 | 0.13 | 95.22 | 3.89 | 0.76 |
| D | Injection 1 | 0.13 | 94.76 | 4.39 | 0.72 |
| 4° C.-7 d | Injection 2 | 0.13 | 94.72 | 4.40 | 0.75 |

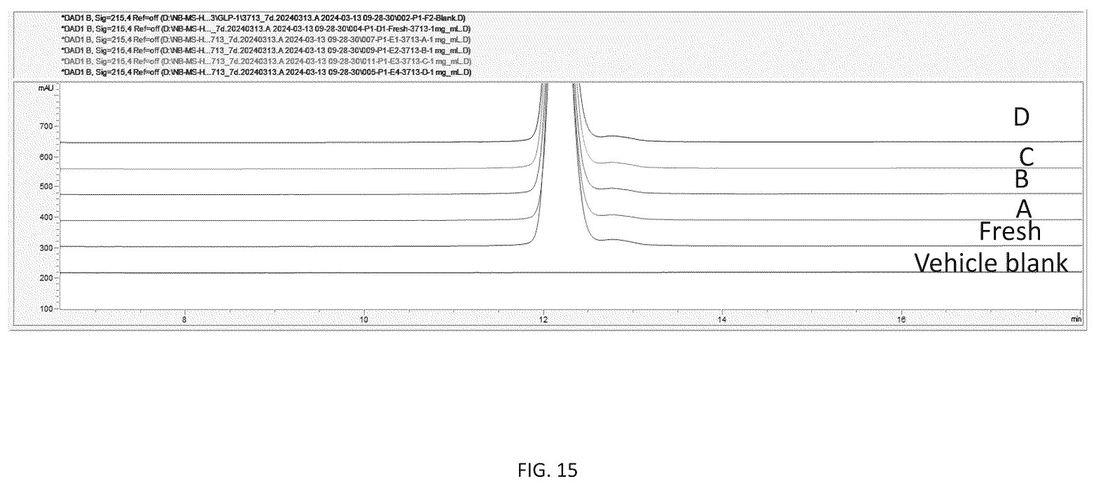

The stability of APi3713 is shown in Table 13 and FIG. 15. No significant change of purity was observed after APi3713 storage at 4° C. or −80° C. for 7 days. The concentration of the formulation remained consistent at 1 mg/mL.

TABLE 13
APi3713 Stability in Formulation

| | | Concentration | | HPLC |
|---|---|---|---|---|
| | | of HPLC | | Purity |
| Compound | Formulation | (mg/mL) | | (%) |
| APi3713 | Fresh | Injection 1 | 1.03 | 97.70 |
| | (Actual = 1.03 mg/mL) | Injection 2 | 1.03 | 97.73 |
| | A | Injection 1 | 0.86 | 97.64 |
| | −80° C.-7 d | Injection 2 | 0.83 | 97.64 |
| | B | Injection 1 | 0.89 | 97.67 |
| | 4° C.-1 d | Injection 2 | 0.89 | 97.71 |
| | C | Injection 1 | 0.94 | 97.67 |
| | 4° C.-3 d | Injection 2 | 0.94 | 97.72 |
| | D | Injection 1 | 0.96 | 97.74 |
| | 4° C.-7 d | Injection 2 | 0.96 | 97.75 |

### Example 5: Anti-Obesity Efficacy Study in Mice

The anti-obesity effects of GLP-1 analogs APi3709, APi3710, APi3711, APi3712, and APi3713 were tested using standard methods. Male Diet-Induced Obese (DIO C57BL/6 J-55 g) mice were fed a diet of 58% fat and high sugar for 12-16 weeks to induce the DIO condition. Mice were treated with 5 nmol/kg of GLP-1 analogs through subcutaneous injection. Normal mice, not receiving the treatment and DIO mice treated with a vehicle (treated with semaglutide on days 1-8, and PBS after day 8) were used as a negative control. Mice treated with 5 nmol/kg of semaglutide were used as a positive control.

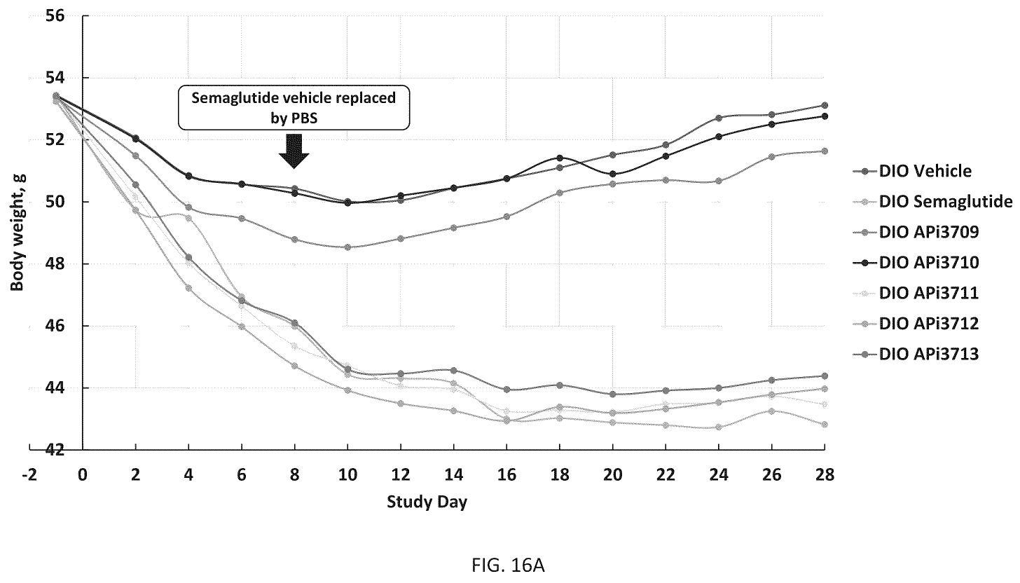

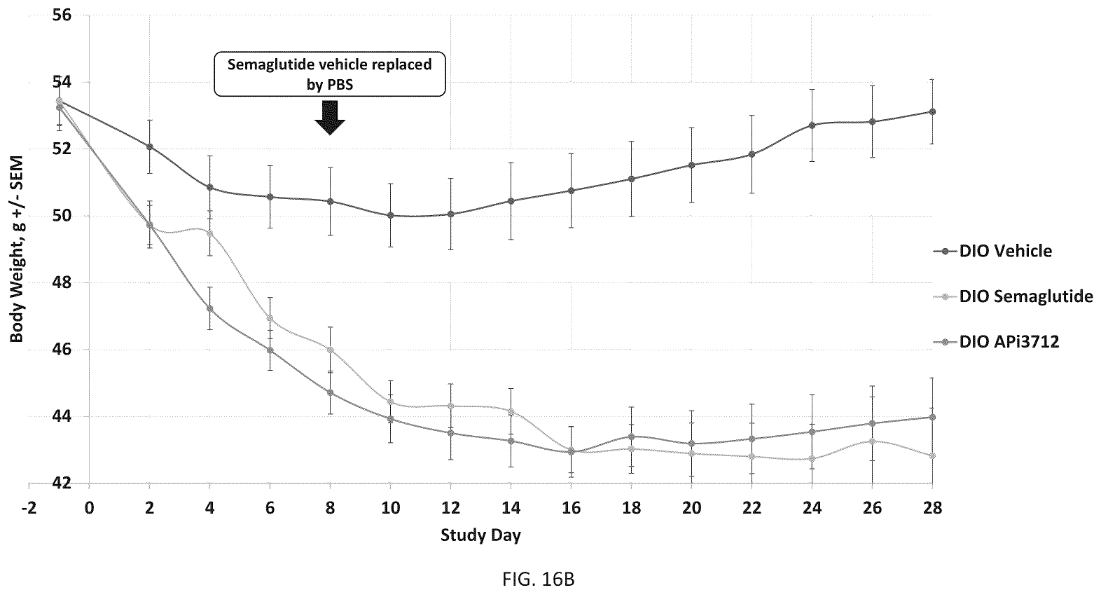

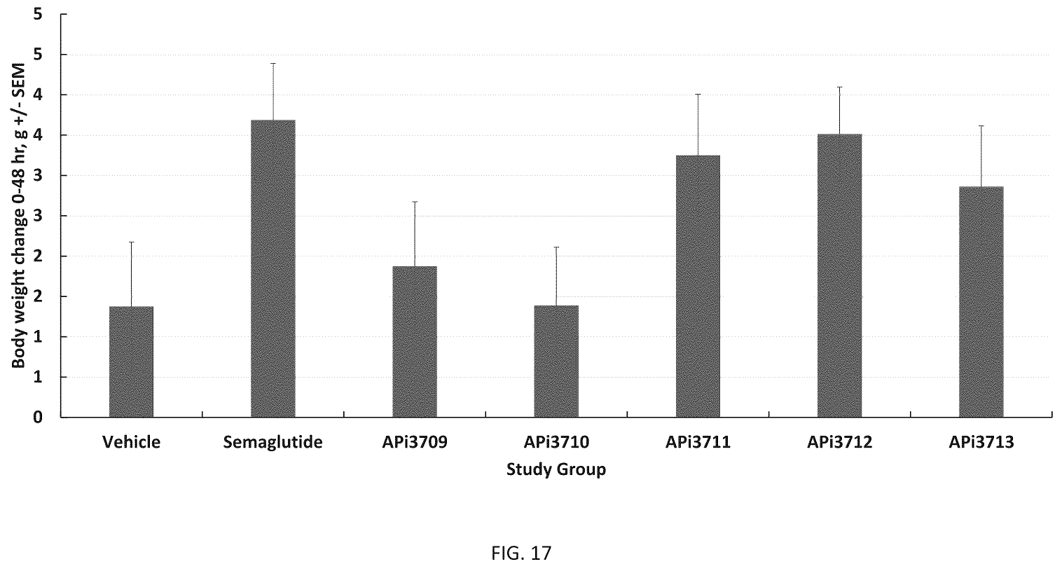

GLP-1 analogs APi3709, APi3710, APi3711, APi3712, and APi3713 demonstrated anti-obesity efficacy similar to semaglutide following 28 days of treatment with 5 nmol/kg subcutaneous injection in DIO mice (FIGS. 16A-16B and 17).

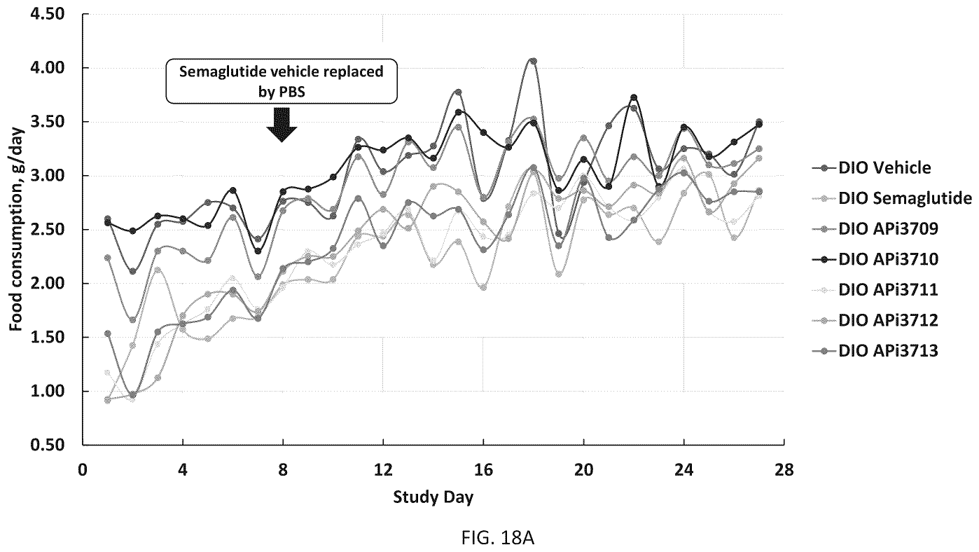

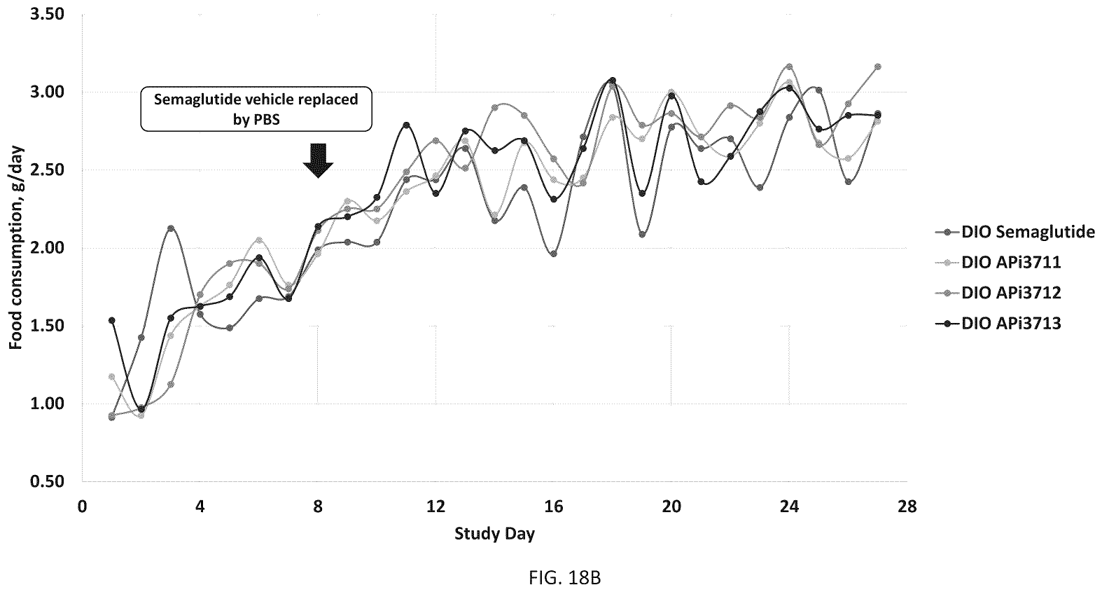

GLP-1 analogs APi3709, APi3710, APi3711, APi3712, and APi3713 demonstrated a food consumption effect similar to semaglutide following 28 days of treatment with 5 nmol/kg subcutaneous injection in DIO mice (FIGS. 18A-18B).

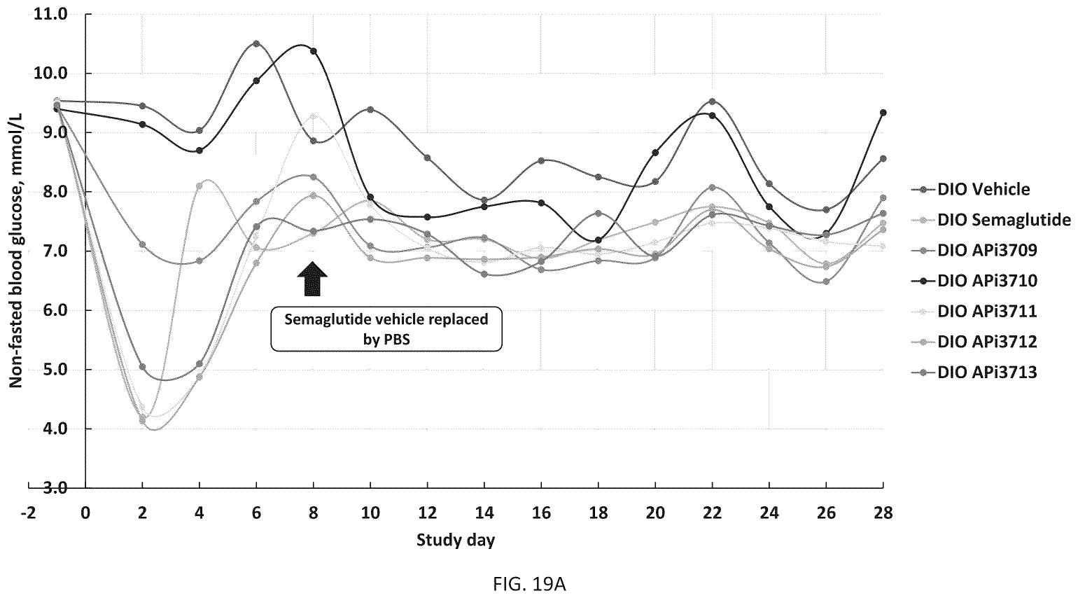

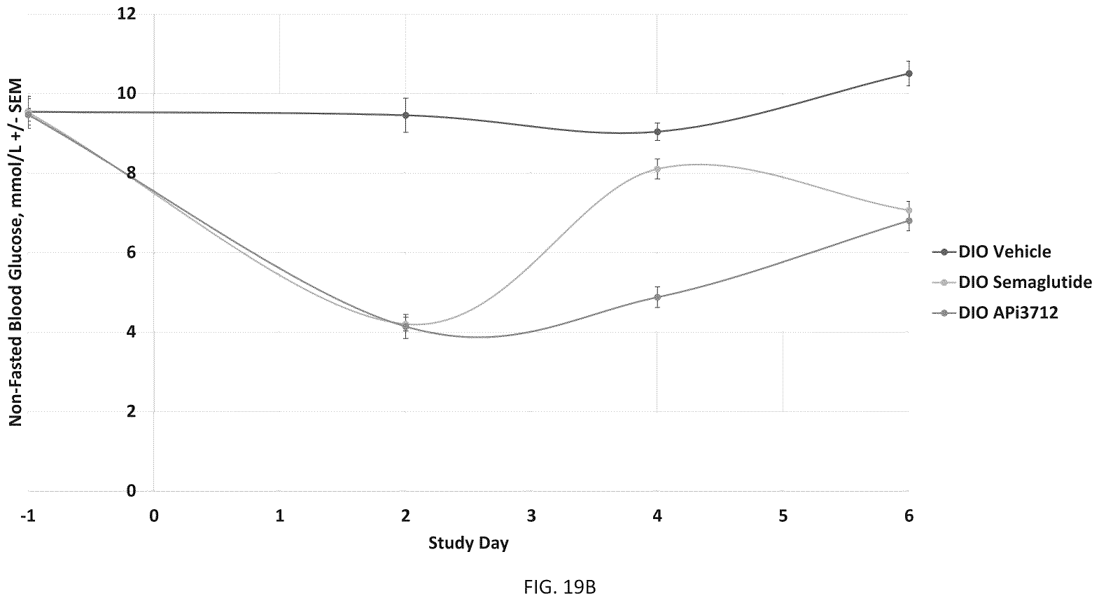

GLP-1 analogs APi3709, APi3710, APi3711, APi3712, and APi3713 demonstrated an effect on non-fasted blood glucose levels similar to semaglutide following 28 days of treatment with 5 nmol/kg subcutaneous injection in DIO mice. (FIGS. 19A-19B).

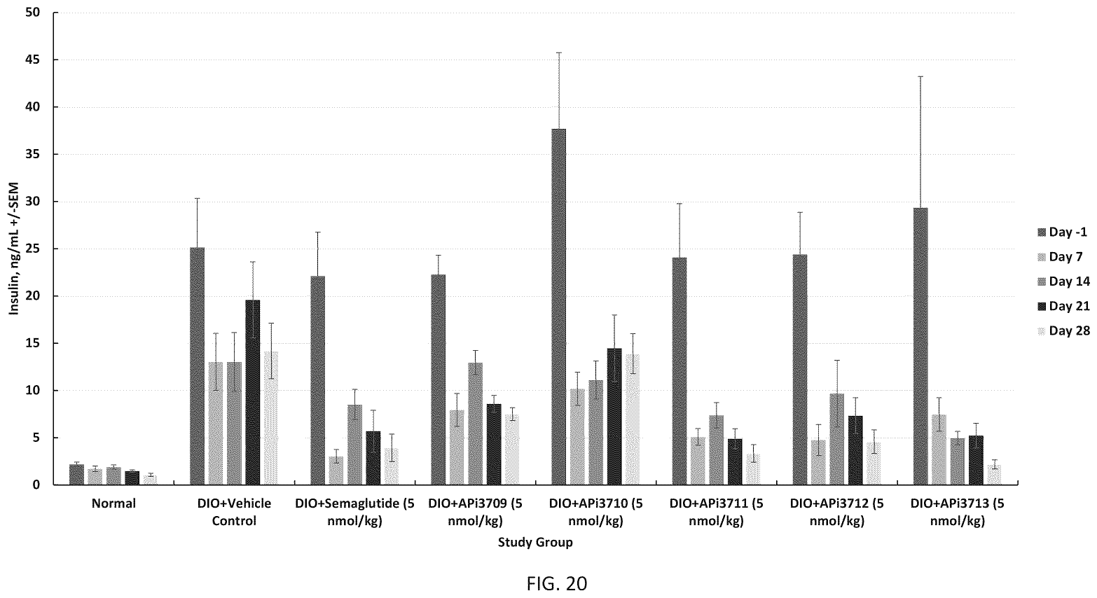

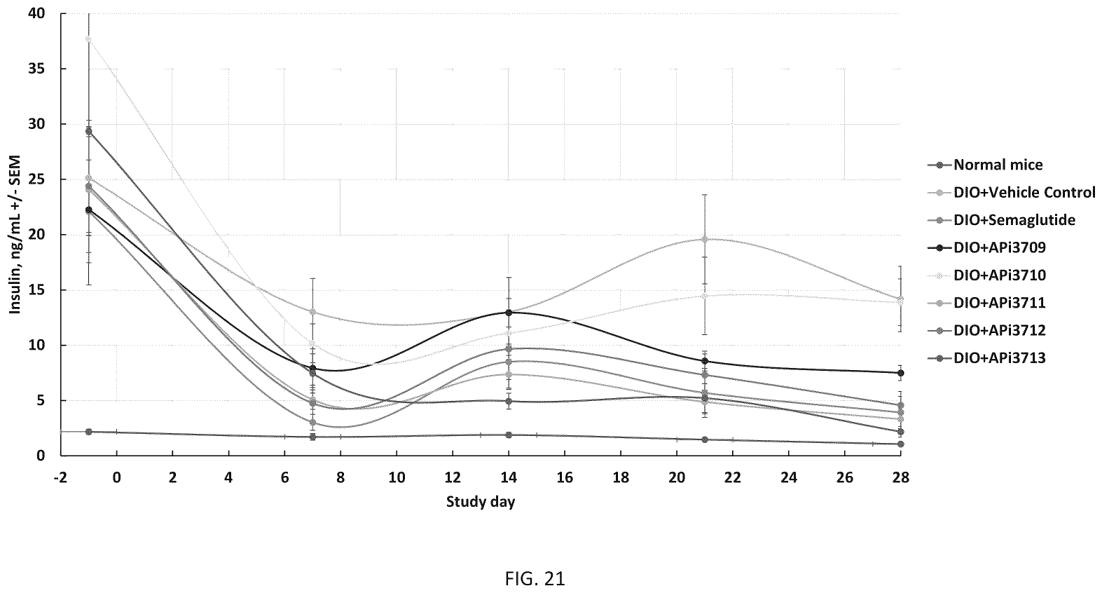

GLP-1 analogs APi3711, APi3712, and APi3713 demonstrated an effect on plasma insulin levels similar to semaglutide following 28 days of treatment with 5 nmol/kg subcutaneous injection in DIO mice. In the APi3713 treated group of mice, insulin levels dropped to normal levels (FIGS. 20 and 21).

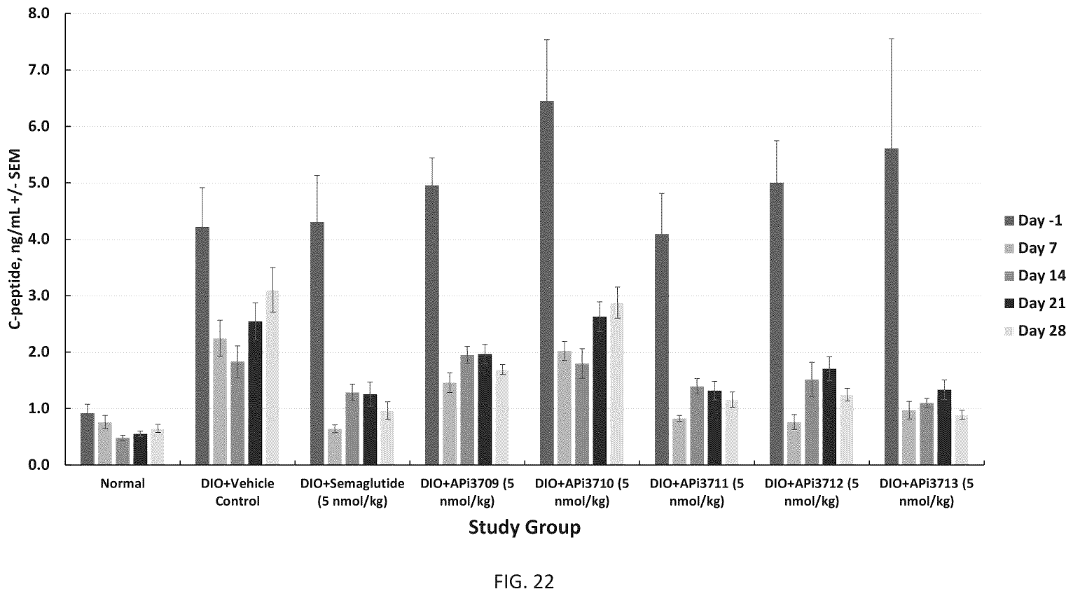

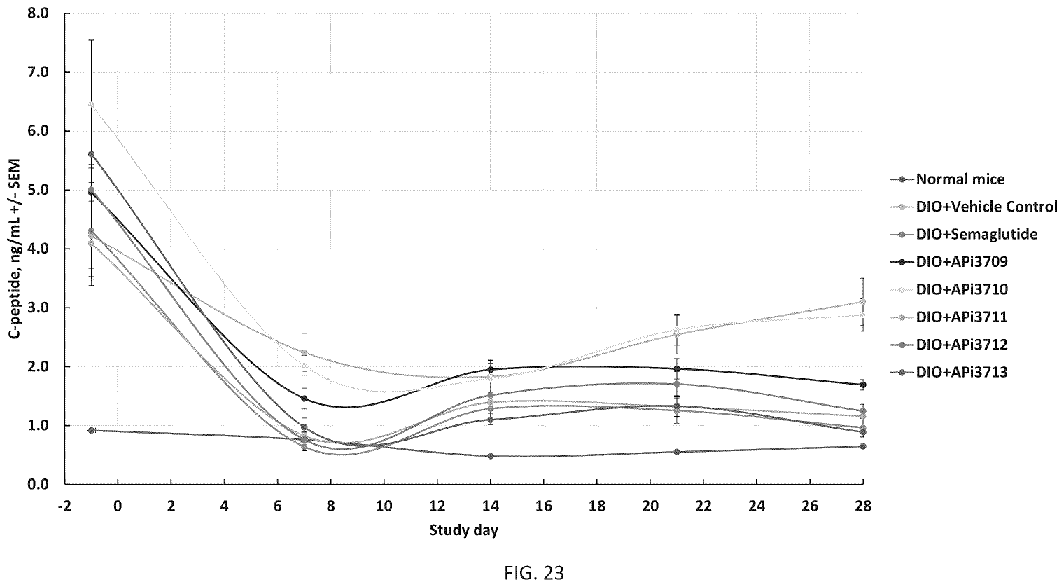

Similarly, GLP-1 analogs APi3709, APi3710, APi3711, APi3712, and APi3713 demonstrated a similar effect on c-peptide plasma concentration that followed the changes in plasma insulin levels (FIGS. 22 and 23).

### Example 6: Enzymatic Stability of GLP-1 Analogs

The enzymatic stability of GLP-1 analogs APi3709, APi3710, APi3711, APi3712, and APi3713 is tested. GLP-1 analogs were spiked into various media comprising dipeptidyl peptidase-4 (DPP-4), or neutral endopeptidase 24.11, or a gastrointestinal enzyme. Peptide stability is confirmed over a series of time points using standard analytical techniques. The GLP-1 analogs demonstrate increased stability as compared to native GLP-1 peptides.

### Example 7: Binding Affinity of GLP-1 Analogs to GIPR

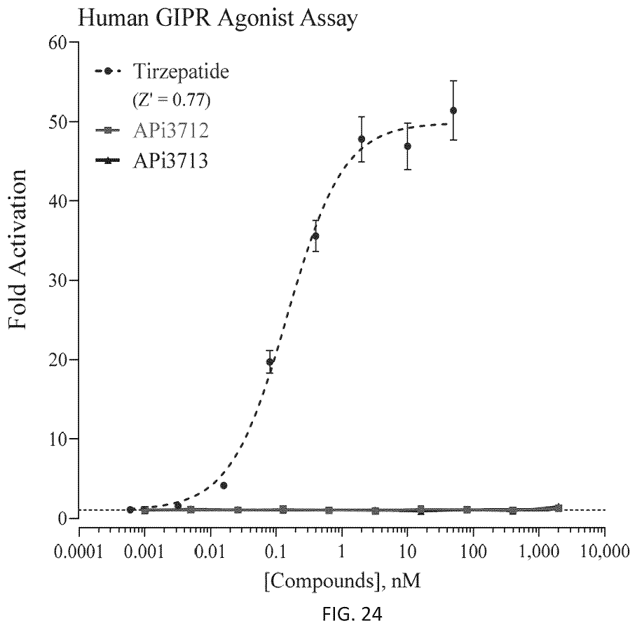

The binding affinity of the GLP-1 analogs APi3712 and APi3713 to GIP-receptor (GIPR) was determined using recombinant Huh7 cells expressing the native GIPR. The reporter gene, firefly luciferase gene was functionally linked and under the control of cAMP response element (CRE) and with constitutive expression of human GIPR. Activation of GIPR in these cells by GLP-1 analog binding was monitored by measuring luciferase activity. The GLP-1 analog binding affinities were compared to the binding affinity of tirzepatide. The results of the binding assay are shown in FIG. 24. APi3713 showed no human GIPR agonist activity despite the presence of the 11 amino acid C-terminal sequence found on the tirzepatide peptide. Similarly, APi3712 showed no GIPR agonist activity.

### EQUIVALENTS

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A GLP-1 analog comprising:
Xaa7-Xaa8-Xaa9-Xaa10-Xa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31-Xaa32-Xaa33-Xaa34-Xaa35-Xaa36-Xaa37 (SEQ ID NO: 1); or
Xaa7-Xaa8-Xaa9-Xaa10-Xa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31-Xaa32-Xaa33-Xaa34-Xaa35-Xaa36-Xaa37-Xaa38-Xaa39-Xaa40-Xaa41-Xaa42-Xaa43-Xaa44 (SEQ ID NO: 2),
wherein
Xaa7 is His or Tyr,
Xaa8 is Ala or Aib or Aib-ψ[CH₂NH],
Xaa9 is Glu,
Xaa10 is Gly,
Xaa11 is Thr,
Xaa12 is Phe,
Xaa13 is Thr,
Xaa14 is Ser,
Xaa15 is Asp,
Xaa16 is Val,
Xaa17 is Ser,
Xaa18 is Ser,
Xaa19 is Tyr or Aib,
Xaa20 is Leu or Aib,
Xaa21 is Glu,
Xaa22 is Gly,
Xaa23 is Gln,
Xaa24 is Ala,
Xaa25 is Ala,
Xaa26 is Lys,
Xaa27 is Glu,
Xaa28 is Phe,
Xaa29 is Ile,
Xaa30 is Ala,
Xaa31 is Trp,
Xaa32 is Leu,
Xaa33 is Val,
Xaa34 is Lys or Arg or Gly,
Xaa35 is Gly,
Xaa36 is Arg or DAP (L-2,3-diaminopropionic acid) or Pro,
Xaa37 is Gly or Ser,
Xaa38 is Ser,
Xaa39 is Gly,
Xaa40 is Ala,
Xaa41 is Pro,
Xaa42 is Pro,
Xaa43 is Pro, and
Xaa44 is Ser.

2. (canceled)

3. The GLP-1 analog of claim 1, wherein the ε-amino group of Lys at position 26 is substituted with a substituent, wherein the substituent is AEEA-AEEA-γ-Glu-(CH₂)ₘ—OH wherein m is 12, 14, 16, 18, 20, or 22.

4. (canceled)

5. The GLP-1 analog of claim 1, wherein the GLP-1 analog is resistant to enzymatic degradation.

6. The GLP-1 analog of claim 5, wherein resistance to enzymatic degradation is determined by comparing the rate of enzymatic degradation of any one of the GLP derivates of claims 1-5 to the rate of enzymatic degradation of any one of native GLP-1 (7-36), native GLP-1(7-37), native GLP-1(7-39), semaglutide, exenatide, lyxumia, dulaglutide, tirzepatide, or liraglutide.

7. The GLP-1 analog of claim 6, wherein the degrading enzyme is a protease or a peptidase.

8. The GLP-1 analog of claim 7, wherein the protease or peptidase is dipeptidyl peptidase-4 (DPP-4), neutral endopeptidase 24.11, or a gastrointestinal enzyme.

9. The GLP-1 analog of claim 1, wherein the GLP-1 analog binding affinity (IC₅₀) to GLP-1 receptor is below 10 nM, below 5 nM, or below 1 nM.

10-11. (canceled)

12. The GLP-1 analog of claim 1, wherein the GLP-1 analog binding affinity is measured by surface plasmon resonance, luciferase activity, a recombinant cell line, or a combination thereof, wherein the recombinant cell line expresses GLP-1 receptor.

13-15. (canceled)

16. The GLP-1 analog of claim 1, wherein the GLP-1 analog exhibits an in vivo plasma elimination half-life of at least 1 hour, or 2 hours, or 4 hours, or 6 hours, or 8 hours, or 10 hours, or 24 hours, or 48 hours, or 72 hours, or 168 hours in human.

17. A pharmaceutical composition comprising a GLP-1 analog of claim 1, and a pharmaceutically acceptable vehicle or carrier.

18. The pharmaceutical composition of claim 17, further comprising an isotonic agent, an excipient, a preservative, a buffer, a surfactant, an anti-diabetic agent, an anti-obesity agent, or a combination thereof.

19-20. (canceled)

21. The pharmaceutical composition of claim 17, wherein the pharmaceutically acceptable carrier is a microsphere, micelle, or nanoparticle.

22. The GLP-1 analog of claim 1, wherein the GLP-1 analog remains stable at a given concentration for several days, weeks, months, or years, at a given temperature.

23. The GLP-1 analog of claim 22, wherein the GLP-1 analog remains stable at a concentration of 1.0 mg/mL for at least 1 day, 2 days, 3 days, 4 days, 5 days, 6 days, or 7 days, at a temperature of −80° C., 4° C., or 8° C.

24. A method of treating diabetes or obesity, comprising administering to a patient a therapeutically effective amount of a GLP-1 analog of claim 1.

25. (canceled)

26. The method of claim 24, wherein the treatment results in the reduction of HbA1C in the patient.

27. The method of claim 24, wherein the treatment results in decreasing food intake, reduction ion body weight, suppression of appetite, inducing satiety in the patient, or a combination thereof.

28. The method of claim 24, wherein the GLP-1 analog is administered by oral, intravenous, parenteral, transdermal, intramuscular, rectal, vaginal, or topical administration.

29-30. (canceled)
